(19) United States
(12) Patent Application Publication
(10) Pub. No.: US 2019/0376925 A1
(43) Pub. Date: Dec. 12, 2019

(54) NUCLEIC ACID SEQUENCING DEVICE CONTAINING GRAPHENE

(71) Applicant: Roswell Biotechnologies, Inc., San Diego, CA (US)

(72) Inventors: Chulmin Choi, San Diego, CA (US); Sungho Jin, San Diego, CA (US); Barry L. Merriman, San Diego, CA (US); Paul Mola, San Diego, CA (US); Tim Geiser, San Diego, CA (US)

(21) Appl. No.: 16/463,195

(22) PCT Filed: Nov. 22, 2017
(86) PCT No.: PCT/US2017/063025

§ 371 Date: May 22, 2019

Related U.S. Application Data
(60) Provisional application No. 62/425,283, filed on Nov. 22, 2016.

(51) Int. Cl. G01N 27/414; B01L 3/00; C12Q 1/6869; G01N 27/30; G03F 7/00

(52) U.S. Cl. CPC G01N 27/4145; B01L 3/502715; C12Q 1/6869; B01L 2300/12; G03F 7/0002; B01L 2300/0645; B01L 2300/0816; G01N 27/308

(57) ABSTRACT

In various aspects of the present disclosure, a sequencing device structure is disclosed. The device structure has an array of metallic conducting electrode pairs, each electrode pair defining a bridging source and drain arrangement separated by a nanogap, the electrode pairs deposited and patterned on a dielectric substrate; a graphene layer deposited onto each electrode pair bridging the source and drain electrodes in each pair, wherein each electrode pair is in electrical isolation from each other; and a dielectric masking layer contacting the graphene layer, the masking layer having an opening exposing a portion of the graphene layer directly over each nanogap, wherein each opening is dimensioned in size to accommodate at least one polymerase enzyme molecule. The graphene layer may include defective graphene.

### CROSS-REFERENCE TO RELATED APPLICATIONS

This application claims priority to U.S. Provisional Patent Application Ser. No. 62/425,283, filed on Nov. 22, 2016, which is incorporated herein by reference in its entirety.

### FIELD

The present disclosure relates generally to biomolecular sensing devices, and more particularly to the fabrication of biomolecular sensing devices for analyzing DNA and related biomolecules in which the biomolecular sensing devices make use of graphene, including defective graphene.

### BACKGROUND

Molecular analysis has received an increasing amount of attention in various fields such as precision medicine or nanotechnology. One example includes the analysis of molecules for sequencing genomes. The seminal work of Maclyn McCarty and Oswald T. Avery in 1946 demonstrated that DNA was the material that determined traits of an organism, (“Studies On The Chemical Nature Of The Substance Inducing Transformation Of Pneumococcal Types II. Effect Of Deoxyribonuclease On The Biological Activity Of The Transforming Substance.”, J. Exp. Med. 83(2), 89-96 (1946)). The molecular structure of DNA was then first described by James D. Watson and Francis HC Crick in 1953 for which they received the 1962 Nobel Prize in Medicine (“Molecular structure of nucleic acids.”, Nature 171,737-738 (1953)). This work made it clear that the sequence of chemical letters (bases) of the DNA molecules encode the fundamental biological information. Since this discovery, there has been a concerted effort to develop means to actually experimentally measure this sequence. The first method for systematically sequencing DNA was introduced by Sanger, et al in 1978, for which he received the 1980 Nobel Prize in Chemistry, (Sanger, Frederick, et al. “The nucleotide sequence of bacteriophage (φ174.” J. Mol. Bio. 125, 225-246 (1978)).

A basic method for sequencing a genome was automated in a commercial instrument platform in the late 1980's, which ultimately enabled the sequencing of the first human genome in 2001. This was the result of a massive public and private effort taking over a decade, at a cost of billions of dollars, and relying on the output of thousands of dedicated DNA sequencing instruments. The success of this effort motivated the development of a number of “massively parallel” sequencing platforms with the goal of dramatically reducing the cost and time required to sequence a human genome. Such massively parallel sequencing platforms generally rely on processing millions to billions of sequencing reactions at the same time in highly miniaturized microfluidic formats. The first of these was invented and commercialized by Jonathan M. Rothberg's group in 2005 as the 454 platform, which achieved thousand fold reductions in cost and instrument time, (e.g., see Marcel Margulies, et al., “Genome Sequencing in Open Microfabricated High Density Picoliter Reactors”, Nature 437, 376-380 (2005) for a review of the process). However, the 454 platform still required approximately a million dollars and took over a month to sequence a genome.

The 454 platform was followed by a variety of other related techniques and commercial platforms, (see, for example: M. L. Metzker, “Sequencing Technologies—the Next Generation”, Nat. Rev. Gen. 11(1), 31-46 (2010); and C. W. Fuller et al, “The Challenges of Sequencing by Synthesis”, Nat. Biotech. 27(11), 1013-1023 (2009)). This progress lead to the realization of the long-sought “$1,000 genome” in 2014, in which the cost of sequencing a human genome at a service lab was reduced to approximately $1,000, and could be performed in several days. However, the highly sophisticated instrument for this sequencing cost nearly one million dollars, and the data was in the form of billions of short reads of approximately 100 bases in length. The billions of short reads often contained errors so the data required interpretation relative to a standard reference genome with each base being sequenced multiple times to assess a new individual genome.

Thus, further improvements in quality and accuracy of sequencing, as well as reductions in cost and time are still needed. This is especially true to make genome sequencing practical for widespread use in precision medicine (see the aforementioned article by Fuller et al), where it is desirable to sequence the genomes of millions of individuals with a clinical grade of quality.

While many DNA sequencing techniques utilize optical means with fluorescence reporters, such methods can be cumbersome, slow in detection speed, and difficult to mass produce to further reduce costs. Label-free DNA or genome sequencing approaches provide advantages of not having to use fluorescent type labeling processes and associated optical systems, especially when combined with electronic signal detection that can be achieved rapidly and in an inexpensive way.

In this regard, certain types of molecular electronic devices can detect single molecule, biomolecular analytes, such as DNAs, RNAs, proteins, and nucleotides, by measuring electronic signal changes when the analyte molecule is attached to a circuit comprising a pair of conductive electrodes. Such methods are label-free and thus avoid using a complicated, bulky and expensive fluorescent type labeling apparatus. These methods can be useful for lower cost sequencing analysis of DNA, RNA and genomes.

While current molecular electronic devices can electronically measure molecules for various applications, they lack the reproducibility as well as scalability and manufacturability needed for rapidly sensing many analytes at a scale of up to millions in a practical manner. Such highly scalable methods are particularly important for DNA sequencing applications, which often need to analyze millions to billions of independent DNA molecules. In addition, the manufacture of current molecular electronic devices is generally costly due to the high level of precision needed.

### SUMMARY

In some embodiments, specially processed, polymerase sensor device structures and methods of manufacture for a multitude of devices usable in electronic DNA, RNA sequencing systems are provided. In some embodiments, these are single-molecule based sequencing analysis systems utilize a defective or nanoporous material component fabricated from graphene, so as to provide advantageous conduction bandgaps, electrical properties, and enhanced attachment of single biomolecules such as polymerase enzyme molecules. In some embodiments, the general electronic sensor system taught here may also be used to detect or analyze other analytes, such as proteins, and disease molecules for diagnostic applications depending on how the molecular sensors are functionalized to interact with various sensing target molecules. The “defective-graphene”-based sequencing systems disclosed herein can be assembled into a massively parallel configuration for rapid sensing and analysis of the interaction of an enzyme with its substrate targets, in particular a polymerase enzyme for applications to sequencing of a DNA molecule, or a collection of such molecules, such as a collection that constitutes an entire human genome.

In various embodiments of the present disclosure, a DNA, RNA or genome sequencing device structure comprises: (a) an array of metallic conducting electrode pairs, each pair defining a source and drain arrangement separated by a nanogap, the metallic conducting electrode pairs being deposited and patterned on a dielectric substrate; (b) a graphene layer deposited onto each electrode pair bridging source and drain electrodes in each pair, leaving each pair in electrical isolation from each other; (c) a dielectric masking layer on the graphene layer, the masking layer having an opening exposing a portion of the graphene layer directly over each nanogap, with each opening dimensioned in size to match the size of a single polymerase enzyme molecule; (d) polymerase enzyme molecules bonded onto each exposed portion of the graphene layer through each opening, with only one molecule per opening; (d) a microfluidic system into which the DNA, RNA or genome sequencing structure (a) through (d) is inserted; and (e) an electronic measurement and computer analysis system by which the incorporation of an analyte onto the polymerase enzyme molecule, as discrete events, are monitored to acquire an electrical signal to determine the identity of the analyte being incorporated, or the nature of other molecular interactions from analysis of the electrical signal. The analytes to be detected include, but are not limited to, nucleotide monomers, deoxynuclosidetriphosphates (dNTPs), DNAs, RNAs, proteins, and the like. The method of detection can be used for lower cost sequencing analysis of DNA, RNA, or entire genomes.

In some embodiments, the graphene layer over each source and drain pair remains suspended over each nanogap without contacting the dielectric layer underneath at the bottom of the nanogap. In some embodiments, the graphene layer over each source and drain electrode pair bridges the source and drain electrodes in each pair and also contacts the dielectric layer within the nanogap. In some embodiments, the nanogap is from about 2 nm to about 20 nm, and each of the metallic conducting electrodes comprise Au, Pt, Ag, Pd, Rh, or alloys therefrom. In certain embodiments, a third electrode is included as a gate electrode.

In various embodiments, the graphene may be single-layer or “few-layer” graphene, and may be pure graphene or defective graphene. Pure graphene layers may be subsequently made defective after the layer of graphene is deposited. Defects include, but are not limited to, linear nano-ribbon parallel array, patterned shape nano-ribbon array, disturbed lattice defects, nanoporous, or compositionally doped defects. In various examples, the defects in the graphene are disturbed lattice defects with a defect density of at least 10⁵/cm², or nanoporous defects having a diameter of at least 2 nm with a defect density of at least 10³/cm². In some instances, the defective graphene has a bandgap opened to a value of at least 0.2 eV or at least 0.5 eV. In other embodiments, the graphene layer is decorated with nanotubes and/or nanowires.

In some embodiments, any of the devices disclosed herein are characterized as having only one polymerase enzyme molecule or other biomolecule per opening over each of the nanogaps between source and drain electrodes. In various embodiments, the one enzyme per opening configuration is preferably at least 80%. Having only one enzyme per opening ensures that each enzyme-analyte interaction or attachment will result in a detectable change in the electrical properties of the molecular bridge. In some embodiments, the polymerase enzyme molecule or other biomolecule are passively bonded to the graphene or defective graphene, (e.g. by weak Van der Waals interactions), or the bonding of biomolecule to graphene may comprise one or more ionic or covalent bonds. In various embodiments, a bifunctional linker is bonded at one end to the graphene via pi-stacking to a pyrene and bonded to the biomolecule at the other end through functional pair conjugation. For example, a bifunctional linker may bond to a polymerase enzyme molecule through functional binding pairs selected from streptavidin-biotin pair, mercaptocarbonic acid [HS-(CH₂)n—COOH, n=1−15] pairs, thiol-alkyne pair, COOH—NH₂ functional group pairs, thiol-maleimide pair, cysteine-maleimide pair, silanization linkage pairings using mercaptosilane compounds, an

NHS (N-hydroxysuccinimide) ester-amine pair, an antigen-antibody pair, or a click chemistry pair.

In various embodiments, the construct comprising the metallic conducting electrode pairs, graphene layer, dielectric masking layer, and enzyme polymerase molecules may be arranged into 2D- or 3D-arrays (e.g. using a stacked microfluidic chamber or a common microfluidic chamber) so as to allow massively parallel electronic sequencing analysis using devices organized into systems having at thousands to millions of individual devices.

In various embodiment of the present disclosure, a method of fabricating a DNA sequencing device structure comprises: (a) depositing and patterning an array of metallic conducting electrode pairs on a dielectric substrate, each pair defining a source and drain arrangement separated by a nanogap; (b) depositing a graphene layer over each metallic conducting electrode pair by using a liquid container lift-up placement method onto the array of metallic conducting electrode pairs, by using a vacuum transfer method, or by using a stamp transfer method, and processing the graphene to obtain a defective graphene material that bridges each source and drain in a pair while leaving each of the electrode pairs in electrical isolation from each other; (c) nanopatterning a dielectric masking layer on the defective graphene material, said masking layer having an opening exposing a portion of said defective graphene material directly over each nanogap, each opening dimensioned in size to match the size of a single polymerase enzyme molecule; (d) placing the construct resulting from steps (a) through (c) into a microfluidic system; and (e) supplying a solution of polymerase enzyme molecules to the microfluidic system to bond a single polymerase enzyme on the exposed portions of said defective graphene material, in the arrangement of one enzyme molecule per opening. In some embodiments, the graphene layer is made defective by nanopatterning into linear nano-ribbon parallel array or patterned shape nano-ribbon array, or by introducing disturbed lattice defects, or by providing nanoporous defects, or by creating compositionally doped defects. In some embodiments, graphene disturbed lattice defects are introduced by beam irradiation selected from ion implantation beam, plasma reactive ion etch (RIE) atmosphere, broadened optical, electron, ion or neutron beam, or scanning beam so as to introduce a defect density of at least 10⁵/cm². In some embodiments, the irradiated structure may be post-irradiation annealed at 100-600° C. In some embodiments, the graphene disturbed lattice defects or nanopores are introduced by chemical etching using oxidizing chemicals, strong acids, strong alkaline solutions, or potassium chlorate-containing solution so as to introduce defect density of at least 10⁵/cm².

In some embodiments, nanoporous defects are introduced by using a nanomask created by irradiating a thin film mask layer with the graphene plasma etched or chemically etched through the mask pores followed by a removal of the thin film mask, so as to introduce a defect density of at least 10⁵/cm². In some embodiments, nanoporous defects are introduced by using block copolymer templated hole generation so as to introduce a defect density of at least 10⁵/cm². In some embodiments, nanoporous defects are introduced by using anodized aluminum oxide templated hole generation so as to introduce a defect density of at least 10⁵/cm². In some embodiments, nanoporous defects are introduced by using nanoimprinting patterning so as to introduce a defect density of at least 10⁵/cm² or by using nanoparticles spray deposited onto the graphene and performing chemical etching or oxidation reactions.

In various embodiments, a method of detecting and identifying an analyte comprises; (a) depositing and patterning an array of metallic conducting electrode pairs on a dielectric substrate, each pair defining a source and drain arrangement separated by a nanogap; (b) depositing a graphene layer over each metallic conducting electrode pair by using a liquid container lift-up placement method onto the array of metallic conducting electrode pairs, by using a vacuum transfer method, or by using a stamp transfer method, and processing the graphene to obtain a defective graphene material that bridges each source and drain in a pair while leaving each of the electrode pairs in electrical isolation from each other; (c) nanopatterning a dielectric masking layer on the defective graphene material, said masking layer having an opening exposing a portion of said defective graphene material directly over each nanogap, each opening dimensioned in size to match the size of a single polymerase enzyme molecule; (d) placing the construct resulting from steps (a) through (c) into a microfluidic system and supplying a solution of polymerase enzyme molecules into the microfluidic system to bond a single polymerase enzyme onto each exposed portion of said defective graphene material, in the configuration of one molecule per opening; (e) supplying a fluid or series of fluids comprising at least one analyte requiring detection; and (f) making an electronic measurement and computer analysis of individual analyte- polymerase molecule interactions and/or incorporations as discrete events, to obtain electrical signals that determine the analyte identity.

In addition to detecting and identifying analytes in general, in some embodiments, the devices in accordance with the present disclosure may be used to diagnose diseases with a genetic component, such as monogenic or polygeneic heritable diseases, somatic mutation disease such as cancer or tissue damage, infectious disease, such as viral or bacterial infection, or microbiome diseases or disorders.

In another aspect, a DNA or genome sequencing device structure is disclosed. The device includes an array of metallic conducting electrode pairs, each pair defining a bridging source and drain arrangement separated by a nanogap, the metallic conducting electrode pairs deposited and patterned on a dielectric substrate; a graphene layer deposited onto each electrode pair bridging source and drain electrodes in each pair, leaving each pair in electrical isolation from each other; a dielectric masking layer on the graphene layer, the masking layer having an opening exposing a portion of the graphene layer directly over each nanogap, each opening dimensioned in size to match the size of a single polymerase enzyme molecule; polymerase enzyme molecules bonded onto each exposed portion of the graphene layer through each of said openings, one molecule per opening; a microfluidic system encasing the DNA or genome sequencing structure described herein; and an electronic measurement and computer analysis system by which the incorporation of a nucleotide monomer, or attachment of a protein, a DNA template or other biomolecular component onto the polymerase enzyme molecule, as discrete events, are monitored to acquire an electrical signal to determine the identity of the nucleotide being incorporated, or the identity of other molecular interactions from analysis of the signal.

In embodiments, the metallic conducting electrode pair comprises at least one of Au, Pt, Ag, Pd, Rh, or their alloys. In embodiments, the metallic conducting electrode pair has a nanogap spacing between the source and drain electrodes of about 2-20 nm. In embodiments, the graphene is defective. In embodiments, the defects are selected from linear nano-ribbon parallel array, patterned shape nano-ribbon array, disturbed lattice defects, nanoporous defects, or compositionally doped defects. In some embodiments, the defects in the graphene are disturbed lattice defects with a defect density of at least 10⁵/cm². In embodiments, the defects in the graphene layer are nanoporous defects having a diameter of at least 2 nm with a defect density of at least 10³/cm². In embodiments, the defective graphene has a bandgap opened to a value of at least 0.2 eV or at least 0.5 eV. In embodiments, the graphene layer is decorated with elongated nanowires selected from carbon nanotubes and semiconductor nanowires of doped Si, Ge, or ZnO. In embodiments, the graphene layer is positioned on each metallic electrode pair defining each nanogap such that the graphene layer is suspended over the substrate in each nanogap and does not contact the substrate in each nanogap. In embodiments, the graphene layer is in contact with the dielectric substrate, with each pair of metallic conducting electrodes extending from both ends of the graphene layer as electrical lead wires. In embodiments, bonding between the polymerase enzyme molecule or polymerase enzyme molecules and the graphene layer comprises van der Waals interactions. In embodiments, bonding between the polymerase enzyme molecule or polymerase enzyme molecules and the graphene layer comprises a bifunctional linker bonded at one end of the linker to the graphene via pi-stacking to a pyrene group. In embodiments, the bifunctional linker bonds to the polymerase enzyme molecule or polymerase enzyme molecules through functional binding pairs selected from streptavidin-biotin pair, mercaptocarbonic acid [HS-(CH₂)n—COOH, n=1-15] pairs, thiol-alkyne pair, COOH-NH₂ functional group pairs, thiol-maleimide pair, cysteine-maleimide pair, silanization linkage pairings using mercaptosilane compounds, an NHS (N-hydroxysuccinimide) ester-amine pair, an antigen-antibody pair, or a click chemistry pair. In embodiments, the structure further includes a gate electrode placed parallel to each source and drain electrodes or placed perpendicular to the nanogap spacing between each source and drain electrodes. In embodiments, the metallic conducting electrodes, graphene layer, dielectric masking layer, and enzyme polymerase molecule are arranged into an array configuration so as to allow massively parallel electronic sequencing analysis using devices organized into a system having at least 1,000 or at least 1 million devices. In embodiments, a sequential interrogation of an array of electrodes in the device is possible for DNA or genome sequencing by using a common lead wire on one side of the array. In embodiments, the metallic conducting electrodes, graphene layer, dielectric masking layer, and polymerase enzyme molecule are arranged into a three-dimensional array using a stacked microfluidic chamber or a common microfluidic chamber for a stacked layer of devices arrays, so as to allow massively parallel electronic sequencing analysis using devices organized into a system having at least 1,000, or at least 1 million devices, or at least 1 billion devices.

In another aspect, a method of fabricating a DNA sequencing device structure is disclosed. The method involves said method depositing and patterning an array of metallic conducting electrode pairs on a dielectric substrate, each pair defining a source and drain arrangement separated by a nanogap; depositing a graphene layer over each metallic conducting electrode pair by using a liquid container lift-up placement method onto the array of metallic conducting electrode pairs, by using a vacuum transfer method, or by using a stamp transfer method, and processing the graphene to obtain a defective graphene material that bridges each source and drain in a pair while leaving each of the electrode pairs in electrical isolation from each other; nanopatterning a dielectric masking layer on the defective graphene material, said masking layer having an opening exposing a portion of said defective graphene material directly over each nanogap, each opening dimensioned in size to match the size of a single polymerase enzyme molecule; placing the construct resulting from prior steps of the disclosed method into a microfluidic system; and supplying a solution of polymerase enzyme molecules to the microfluidic system to bond a single polymerase enzyme on the exposed portions of said defective graphene material, in the arrangement of one enzyme molecule per opening.

In embodiments, the graphene layer is made defective by nanopatterning into linear nano-ribbon parallel array or patterned shape nano-ribbon array, or by introducing disturbed lattice defects, or by providing nanoporous defects. In embodiments, the graphene disturbed lattice defects are introduced by beam irradiation selected from ion implantation beam, plasma reactive ion etch (RIE) atmosphere, broadened optical, electron, ion or neutron beam, so as to introduce a defect density of at least 10⁵/cm². In embodiments, the irradiated structure is post-irradiation annealed at 100-600° C. In embodiments, the graphene disturbed lattice defects or nanopores are introduced by chemical etching using oxidizing chemicals, strong acids, strong alkaline solutions, or potassium chlorate-containing solution so as to introduce defect density of at least 10⁵/cm². In embodiments, the nanoporous defects are introduced by using block copolymer templated hole generation so as to introduce a defect density of at least 10⁵/cm². In embodiments, nanopatterning the defective graphene involves the steps of: preparing a defective thin film mask layer having a thickness of 0.2-5 nm on the surface of the graphene layer by sputtering or evaporation deposition of a material selected from metallic, ceramic or polymeric layer, wherein the thin film mask comprise pinholes with a density of at least 10⁵/cm, wherein the average diameter of the pinholes being in the range of 0.2-5 nm; applying plasma etching methods to introduce defects in the graphene layer of holes to produce hole-shaped defects and cavities with a density of at least 10⁵/cm, wherein the average diameter of the hole-shaped defects are in the range of 0.2-5 nm; and removing the thin film mask on the surface of the graphene layer by chemical or physical etching or dissolving. In embodiments, the nanoporous defects are introduced by using nanoimprinting patterning so as to introduce a defect density of at least 10⁵/cm². In embodiments, the nanoporous defects are introduced by using nanoparticles spray deposited onto the graphene and performing chemical etching or oxidation reactions. In another aspect, a method is disclosed of improving adhesion of polymerase type enzyme molecules or other biomolecules onto a surface of graphene; and applying a bias voltage of plus/minus 50-100 volts. In embodiments, the device disclosed herein is used to perform whole genome sequencing. When the device disclosed herein is not in use, the device is back-filled with inert gas so as to minimize inadvertent adhesion or adsorption of unwanted molecules in the device.

In another aspect, the devices disclosed herein are used to diagnose diseases with a genetic component, such as monogenic or polygeneic heritable diseases, somatic mutation disease such as cancer or tissue damage, infectious disease, such as viral or bacterial infection, or microbiome diseases or disorders. In embodiments, the electrode structures and supporting measurement circuitry as disclosed herein are all embodied in pixels of a sensor array, which in turn is embodied in a semiconductor chip made via CMOS processing.

In another aspect, a method of detecting and identifying an analyte is disclosed. The method involves depositing and patterning an array of metallic conducting electrode pairs on a dielectric substrate, each pair defining a source and drain arrangement separated by a nanogap; depositing a graphene layer over each metallic conducting electrode pair by using a liquid container lift-up placement method onto the array of metallic conducting electrode pairs, by using a vacuum transfer method, or by using a stamp transfer method, and processing the graphene to obtain a defective graphene material that bridges each source and drain in a pair while leaving each of the electrode pairs in electrical isolation from each other; nanopatterning a dielectric masking layer on the defective graphene material, said masking layer having an opening exposing a portion of said defective graphene material directly over each nanogap, each opening dimensioned in size to match the size of a single polymerase enzyme molecule; placing the construct resulting from steps detailed herein into a microfluidic system and supplying a solution of polymerase enzyme molecules into the microfluidic system to bond a single polymerase enzyme onto each exposed portion of said defective graphene material, in the configuration of one molecule per opening; supplying a fluid or series of fluids comprising at least one analyte requiring detection; and making an electronic measurement and computer analysis of individual analyte- polymerase molecule interactions and/or incorporations as discrete events, to obtain electrical signals that determine the analyte identity. In embodiments, the analyte comprises DNA template molecules and substrates for nucleotide incorporation. In embodiments, the concept of analyte identity comprises specific nucleotide base identity and a sequence of nucleotide incorporations by said polymerase enzyme molecules.

In aspects of the present disclosure, and concerning the sequencing device structure detailed herein, the dNTP nucleotide being attached to the enzyme polymerase molecule is a modified nucleotide type to enhance the incorporation signals, or produce signals with enhanced differences between the different base (A, C, G, T) incorporation events, for greater accuracy determining the template sequence, with such dNTP modifications including modifications of: the base, such as 7-deaza forms, 8-bromo forms, or the alpha- and beta-phosphates, such as thiolated forms or bromated forms of these phosphates, or gamma-phosphate modifications, including the addition of phosphates, such as tetra-, penta- or hexa-phosphates forms, or the groups added to the terminal phosphate. In further aspects of the present disclosure, a method is disclosed in which modified dNTPs are attached to the polymerase enzyme. The modified dNTPs include modifications selected from: modifying the base, such as 7-deaza forms, 8-bromo forms; modifying alpha-and beta- phosphates, such as thiolated forms or bromated forms of these phosphates; modifying the gamma-phosphate modifications, including the addition of phosphates, such as tetra-, penta- or hexa- phosphate forms; and modifying the groups added to the terminal phosphates. In embodiments, the modified dNTPs function to enhance incorporation signals. In embodiments, the modified dNTPs function to produce signals with enhanced differences between incorporation of the different nucleotides A, T, C, and G, wherein the enhanced differences between incorporation of the different nucleotides A, T, C, and G results in greater accuracy in determining the template sequence.

In another aspect of the present disclosure, a sequencing device structure is disclosed. The device structure includes an array of metallic conducting electrode pairs, each electrode pair defining a bridging source and drain arrangement separated by a nanogap, the electrode pairs deposited and patterned on a dielectric substrate; a graphene layer deposited onto each electrode pair bridging the source and drain electrodes in each pair, wherein each electrode pair is in electrical isolation from each other; and a dielectric masking layer contacting the graphene layer, the masking layer having an opening exposing a portion of the graphene layer directly over each nanogap, wherein each opening is dimensioned in size to accommodate at least one polymerase enzyme molecule. In embodiments, the sequencing device structure further includes at least one polymerase enzyme molecule bonded to the exposed portion of the graphene layer through the opening. In embodiments, the sequencing device structure further includes a microfluidic system in fluid combination with the sequencing device structure to provide the at least one polymerase enzyme molecule. In embodiments, each electrode pair comprises at least one of Au, Pt, Ag, Pd, Rh, or their alloys. In embodiments, the nanogap is about 2 nm to about 20 nm in length. In embodiments, the graphene layer comprises defective graphene. In embodiments, the defective graphene comprises a linear nano-ribbon parallel array, a patterned shape nano-ribbon array, disturbed lattice defects, nanoporous defects, or compositionally doped defects. In embodiments, the defective graphene comprises disturbed lattice defects having a defect density of at least about 10⁵/cm². In embodiments, the defective graphene comprises nanoporous defects having a diameter of at least about 2 nm with a defect density of at least about 10³/cm². In embodiments, the defective graphene has a bandgap opened to a value of at least 0.2 eV. In embodiments, the defective graphene comprises elongated nanowires that are selected from carbon nanotubes and semiconductor nanowires of doped Si, Ge, or ZnO. In embodiments, the graphene layer is positioned on each electrode pair such that the graphene layer does not contact the substrate at each nanogap. In embodiments, the bonding of the at least one polymerase enzyme to the graphene layer comprises van der Waals interactions. In embodiments, the bonding between the polymerase enzyme molecule and the graphene layer comprises a bifunctional linker bonded at one end of the linker to the graphene layer by pi-stacking to a pyrene group. In embodiments, the bifunctional linker bonds to the polymerase enzyme molecule through functional binding pairs selected from streptavidin-biotin pair, mercaptocarbonic acid [HS-(CH₂)n-COOH, n=1-15] pairs, thiol-alkyne pair, COOH-NH₂ functional group pairs, thiol-maleimide pair, cysteine-maleimide pair, silanization linkage pairings using mercaptosilane compounds, an NHS (N-hydroxysuccinimide) ester-amine pair, an antigen-antibody pair, or a click chemistry pair. In embodiments, the sequencing device structure further includes a gate electrode that is parallel to each electrode pair defining a bridging source and drain arrangement. In embodiments, the sequencing device structure further includes a gate electrode that is perpendicular to the nanogap spacing between each electrode pair defining a bridging source and drain arrangement.

In another aspect of the present disclosure, a method of fabricating a sequencing device structure is disclosed. The method involves depositing and patterning an array of metallic conducting electrode pairs on a dielectric substrate, each electrode pair defining a source and drain arrangement separated by a nanogap; depositing a graphene layer over each metallic conducting electrode pair; and nanopatterning a dielectric masking layer on the graphene layer. In embodiments, the method further involves processing the graphene layer to obtain a defective graphene material. In embodiments, the processing involves nanopatterning the graphene layer into a linear nano-ribbon parallel array or a patterned shape nano-ribbon array, introducing disturbed lattice defects, or providing nanoporous defects.

### BRIEF DESCRIPTION OF THE DRAWINGS

The features and advantages of the embodiments of the present disclosure will become more apparent from the detailed description set forth below when taken in conjunction with the drawings. The drawings and the associated descriptions are provided to illustrate embodiments of the disclosure and not to limit the scope of what is claimed. FIGS. 1a-d illustrate exemplary defects that may be artificially introduced to a graphene sheet to provide an increased bandgap and also to provide many active site edge locations for strong adhesion of bridge structures or biomolecules (such as enzyme molecules). The graphene can be of armchair type, zig-zag type or chiral type.

FIGS. 2a-d illustrate fabrication steps for an exemplary molecular bridge of DNA or RNA sequencing sensor comprising size-limited, graphene islands: FIG. 2a details depositing and patterning conducting electrode pair (source and drain); FIG. 2b details placing a suspended graphene sheet thereon; FIG. 2c details adding protective dielectric coating; and FIG. 2d details allowing a single molecule enzyme (polymerase) to attach onto the graphene, and enable each of the polymerase reactions of nucleotide incorporation (nucleotide monomers like A, T, C, G, U, etc., incorporated by the polymerase reacting with their respective deoxynucleosidetriphosphate (dNTP) forms provided in solution) to change electrical properties of the molecular bridge for sequencing analysis.

FIGS. 3a-c illustrate exemplary embodiments of two configurations of graphene bridge placement: FIG. 3a details a suspended graphene bridge; FIG. 3b details a substrate-adhered graphene bridge; and FIG. 3c is a top view of the graphene bridge structure with an attached polymerase enzyme. The single molecule enzyme (polymerase) on graphene enables each of the polymerase reaction of nucleotide incorporation (nucleotide monomers like A, T, C, G, U, etc. incorporated by the polymerase reacting with their respective deoxynucleosidetriphosphate (dNTP) forms provided in solution) to change electrical properties of the molecular bridge for sequencing analysis.

FIGS. 4a-b illustrates enzyme molecule connection to graphene-derived material via functional groups, with two exemplary embodiments of solid state molecular sensor, based on a suspended graphene bridge (shown in FIG. 4a); and a substrate-adhered graphene bridge (as shown in FIG. 4b). In various embodiments, a defective or porous graphene layer is utilized, which is masked to a limited size area (e.g., 2-20 nm) to enforce a single molecule attachment using a patterned dielectric coating (e.g., PMMA, PDMS or other adhesion-impeding polymer layer or SiO₂ layer). The connector may be a bifunctional linker, such as one that comprises a first group of pyrene for pi-stack binding, and second group such as one from maleimide, biotin, NHS, an amine group, or a “click chemistry” group, such as DBCO.

FIGS. 5a-b illustrate embodiments of a gated structure of the graphene bridge sequencing device with the gate electrode placed as: parallel to the source and drain electrodes (as shown in FIG. 5a); or perpendicular to the nanogap spacing between the two (source and drain) electrodes (as shown in FIG. 5b). The tip of the gate electrode can also be placed above or below the illustrated nanogap. Such a gate electrode may also be buried below the surface, as a so-called “backgate,” (not shown).

FIG. 6 illustrates a top view of an array of graphene bridge molecular sensors, with the graphene positioned as a suspended layer or substrate-adhered layer. The polymerase enzyme molecule is shown only on the lowest electrode pair. Massively parallel electronic sequencing analysis can be performed with many devices organized into a system, having as many as 10,000, or 1 million or 10 million devices.

FIG. 7a illustrates a side view of a molecular bridge solid state sensor using nanogapped graphene-derivative for genome or DNA sequencing detection. Change of current or other electrical signals are measured upon incorporation of nucleotide or attachment or detachment of other biomolecules at the split-graphene tunnel junction configuration. FIG. 7b illustrates a top view of a single enzyme molecule seated and edge-adhered onto a nano-gap graphene-derivative. FIG. 7c illustrates a single enzyme molecule seated and adhered onto a round or oval hole shaped nano-gap edge of graphene. FIG. 7d illustrates a single enzyme molecule well-adhered onto the edge of a round or oval hole intentionally made in graphene.

FIGS. 8a-f illustrate exemplary fabrication steps for a nanopatterned graphene bridge using diblock copolymer mask, comprising: CVD growing of graphene on Cu foil, (forming the starting material) (as shown in FIG. 8a); after Cu was etched 1M FeCl₃, transferring the graphene on SiO₂ (or SiO₂-coated Si), or Au-electrode-pair deposited SiO₂ to collect graphene as a suspended bridge (as shown in FIG. 8b); covering the graphene by a thin layer of evaporated SiO₂ and a thin film of spin-coated block-copolymer PS-b-P4VP (as shown in FIG. 8c); annealing and developing the PS-b-P4VP block-copolymer film into a nanoscale two-phase structure, leaving the porous PS matrix as the template for subsequent patterning (as shown in FIG. 8d); fluoride-based reactive ion etching (RIE) to penetrate and pattern the SiO₂ oxide layer, partially degrading the PS film, and form the hard mask hole pattern out of the thin SiO₂ (as shown in FIG. 8e); etching an exposed hole in the graphene by O₂ plasma, followed by SiO₂ removal (as shown in FIG. 8f). Finally, nanoporous graphene on SiO₂ was obtained, which is then patterned into a limited size island graphene by dielectric masking for single molecule (e.g., enzyme) attachment. Further, FIGS. 8g and 8h are SEM micrographs showing the process of nano patterning of graphene using diblock copolymer mask. FIG. 8g is a micrograph of the phase decomposed block-copolymer film (hexagonally distributed P4VP domains in the PS matrix) on graphene. FIG. 8h is an SEM image of the nanopatterned graphene surface after SiO₂ mask patterning, graphene removal from the mask hole regions, and etching away of SiO₂ mask. The nanopores are ˜30 nm diameter in this embodiment. The diblock copolymer masked patterning approach can produce ˜10-50 nm size defects on graphene.

FIGS. 9a-f illustrate AAO membrane based nanopatterning of graphene for molecular bridge sensors for DNA sequencing. FIG. 9a illustrates CVD grown graphene; FIG. 9b details placement of graphene on a substrate; FIG. 9c details AAO nanohole membrane fabrication; FIG. 9d details placement of AAO on a graphene surface; FIG. 9e details RIE etching of graphene through the AAO holes, and FIG. 9f details nano patterned graphene on substrate obtained after AAO mask removal.

FIGS. 10a-e illustrate in a schematic form the evolution of a thin film structure during deposition, as detailed herein.

FIGS. 11a-d illustrate exemplary perforated and defective graphene for molecular bridge DNA or RNA sequencing sensors. FIG. 11a illustrates the beginning graphene layer; FIG. 11b illustrates coating the graphene surface with nanoparticles (NPs) of ceramic or metal; FIG. 11c illustrates induction of oxidation or degradation reactions by heating followed by etching removal of reacted NPs to obtain perforated graphene; and FIG. 11d illustrates the addition of electrical lead wires and size-limiting dielectric layer (polymer or ceramic) to make the solid state electronic sensor for single enzyme molecule sequencing analysis and sensing the attachment or detachment of related molecular components or targets.

FIGS. 12a-d illustrate defective graphene obtained by a beam irradiation for enhanced molecular bridge DNA or RNA sensors. FIG. 12a illustrates the beginning graphene layer on the substrate; FIG. 12b illustrates irradiation of the graphene with ion implantation beam, plasma reactive ion etch (RIE) atmosphere, broadened optical, electron, ion or neutron beam; FIG. 12c illustrates induction of defective graphene (with optional addition of post-irradiation annealing); and FIG. 12d illustrates addition of electrical lead wires and size-limiting dielectric mask layer (polymer or ceramic) to make the solid state electronic sensor for single enzyme molecule and associated analytes attachment and sequencing analysis. The desired density of defects in graphene is at least 10⁵/cm², preferably at least 10⁷/cm².

FIGS. 13a-d illustrate chemically induced defective graphene (disturbed lattice regions or nanopores). FIG. 13a illustrates the graphene layer; FIG. 13b illustrates chemical etching of graphene surface; FIG. 13c illustrates defective graphene (with optional addition of post-etch annealing); and FIG. 13d illustrates addition of electrical lead wires and size-limiting dielectric layer (polymer or ceramic) to make the solid state electronic sensor for single enzyme molecule and associated analytes attachment and sequencing analysis.

FIGS. 14a-c illustrate combination structures of nanowires (e.g., carbon nanotubes, semiconductor nanotubes or nanowires) adhered onto the surface for defective graphene for enhanced molecular bridge DNA sequencing sensor. FIG. 14a illustrates the graphene layer; FIG. 14b illustrates semiconductor nanotubes or nanowires added on graphene; and FIG. 14c illustrates addition of electrical lead wires and size-limiting dielectric mask layer (polymer or ceramic) to make the solid state electronic sensor for single enzyme molecule and associated analytes attachment and sequencing analysis. The desired density of nanotubes or nanowires on graphene is at least 10³/cm², preferably at least 10⁵/cm² or more. The adherent structures may improve the integrity or modify the electrical properties of the defective graphene.

FIG. 15 illustrates a top view of an array of molecular bridge sensors having a semiconductor nanotube- or nanowire- decorated graphene, with the graphene positioned as a suspended layer or substrate-adhered layer. A single polymerase enzyme molecule placed on top of nanotube- or nanowire-decorated graphene is also illustrated on the nanogap near the lower electrode pair in the drawing.

FIG. 16a illustrates a massively parallel array of solid state molecular sensor device comprising many graphene-containing enzyme polymerase structures for DNA or genome sequencing. An areal distribution of the devices with associate routing of electrical wiring is provided. FIG. 16b illustrates a microfluidic chamber into which enzyme or other biomolecules are supplied in a phosphate-buffered saline (PBS) or other liquid buffer solutions. Individual single enzyme molecules are selectively attached with a high probability onto each of the size-limited, exposed graphene island regions on the massively parallel sensor array. The unused biomolecules which did not adhere to the graphene islands are washed away out of the fluidic chamber. The analytes to be detected such as nucleotides or proteins are then supplied into the chamber to attach to the polymerase enzyme and thereby to induce electrical signals for sequencing analysis.

FIG. 17 illustrates sequential interrogation of electrodes from the graphene-containing enzyme polymerase molecular sensor for DNA or genome sequencing by using a common lead wire on one side of the array.

FIGS. 18a-b illustrate a three-dimensional array of molecular electronics genome-sequencing platform comprising graphene-derived material and polymerase enzyme structure. An electrically insulating top coating (not shown) such as polymer or oxide layer (e.g., aluminum oxide, Si oxide, etc.) is applied except the exposed region of graphene-derived material for polymerase enzyme structure attachment. FIG. 18a illustrates that each two-dimensional molecular electronics genome-sequencing device array is separately packaged into an individual microfluidic system, with each of the microfluidic layer stacked into a three-dimensional system; and FIG. 18b illustrates two-dimensional molecular electronics genome-sequencing device arrays stacked into three dimensional configuration but housed in a single microfluidic system. The total number of molecular sensors can be at least 1,000, and can be as many as 1 million or more for massive parallel genome sequencing analysis.

FIG. 19 illustrates a flow diagram of a DNA or genome sequencing system comprising graphene-derived material and polymerase enzyme sensor array, microfluidic structures, signal detection electronic circuits and associated devices, and data analysis, storage or transmission devices. The system can be embodied in the format of a benchtop unit, portable unit, a hand-held unit, or wearable unit.

It is to be understood that the drawings are for purposes of illustrating the concepts of the embodiments disclosed herein and are not to scale.

### DETAILED DESCRIPTION

In various embodiments of the present disclosure, a DNA or RNA sequencing device structure is described, comprising a graphene-derived material frame with a polymerase enzyme for generation of electronic signals when an individual nucleotide is incorporated into a nucleic acid polymer template via the action of the polymerase. In some embodiments, specially processed, defective or nanoporous graphene is employed so as to utilize altered bandgaps and enhanced attachment of single biomolecules. In some embodiments, the defective-graphene-based sequencing systems invented here is assembled into a massively parallel configuration for rapid analysis of targets including nucleotides, in particular for applications for sequencing of a DNA molecule, or a collection of such molecules constituting an entire genome.

### Definitions

As used herein, the term “bandgap” means the energy range in a solid in which electrons do not exist.

As used herein, the term “graphene” means a single or few layer of carbon atoms arranged in a 2d lattice structure.

As used herein, the term “defective graphene” means graphene having circular, square, rectangle or irregular-shaped defects. The defects can be lattice defects such as atomic vacancies, interstitials, dislocations or distorted lattice planes, or defects that have a somewhat larger dimension than lattice defects, such as holes or inserted aggregates of foreign atom species, or compositionally doped regions by one or more other atomic species such as nitrogen, sulfur, or metallic elements such as chemisorbed or adsorbed Co, Ni, Pd, Mn, Fe, Al, Ag, Cu, Au, and Pt. The defects can result from non-sp² oribital hybrid carbon atoms in the graphene or from non-carbon atoms in the graphene. The defects can be introduced by irradiation with ion beam, neutron beam, electron beam, or laser beam, or by plasma treatment. Such irradiation can also be utilized to prepare a nanoscale or subnanometer scale mask on a metallic, ceramic or polymeric thin film deposited on the surface of graphene, through which the graphene underneath can be locally etched to produce a random defective structure. Larger size defects can also be introduced by lithographic patterning such as e-beam lithography, nanoimprinting lithography, or template-mask induced removal such as using pre-patterned diblock co-polymer membrane, anodized aluminum oxide membrane followed by plasma (or RIE) etching or ion beam etching to remove graphene material followed by mask removal.

As used herein, the term “nucleotide” means either the native dNTPs like A, T, C, G (i.e., dATP, dTTP, dCTP and dGTP), or collectively refers to various types of modified dNTPs. As used herein, the term “polymerase” means an enzyme that synthesizes polymers of nucleic acids. DNA polymerase synthesizes polymers of DNA molecules. RNA polymerase synthesizes polymers of RNA molecules.

As used herein, the term “van der Waals” means a residual attractive between molecules or atomic groups that are not the result of a covalent bond or electrostatic interactions

### Devices

In some embodiments, a DNA or genome sequencing device structure is provided comprising: (a) an array of metallic conducting electrode pairs, each pair defining a bridging source and drain arrangement separated by a nanogap, said metallic conducting electrode pairs deposited and patterned on a dielectric substrate; (b) a graphene layer deposited onto each electrode pair bridging source and drain electrodes in each pair, leaving each pair in electrical isolation from each other; (c) a dielectric masking layer on the graphene layer, said masking layer having an opening exposing a portion of said graphene layer directly over each nanogap, each opening dimensioned in size to match the size of a single polymerase enzyme molecule; (d) polymerase enzyme molecules bonded onto each exposed portion of the graphene layer through each of said openings, one molecule per opening; (d) a microfluidic system encasing the DNA or genome sequencing structure (a) through (d); and (e) an electronic measurement and computer analysis system by which the incorporation of a nucleotide monomer, or attachment of a protein, a DNA template or other biomolecular component onto the polymerase enzyme molecule, as discrete events, are monitored to acquire an electrical signal to determine the identity of the nucleotide being incorporated, or the identity of other molecular interactions from analysis of the signal.

In some embodiments, the conducting electrode pair comprises at least one of Au, Pt, Ag, Pd, Rh, and their alloys.

In some embodiments, each metallic conducting electrode pair has a nanogap spacing between source and drain electrodes of about 0.1-200 nm, for example, between 0.5 and 150 nm, between 1 and 100 nm, between 5 and 50 nm, or between 10 and 25 nm. In some embodiments, each metallic conducting electrode pair has a nanogap spacing between source and drain electrodes of about 2-20 nm.

In some embodiments, the graphene is defective, said defects selected from linear nano-ribbon parallel array, patterned shape nano-ribbon array, disturbed lattice defects, or nanoporous defects. In some embodiments, the graphene is defective due to intrinsic defects resulting from non-sp² orbital hybrid carbon atoms in graphene. In some embodiments, the intrinsic defect is a Stone-Wales defect. In some embodiments, the intrinsic defect is a single vacancy defect. In some embodiments, the intrinsic defect is a multiple vacancy defect. In some embodiments, the intrinsic defect is a line defect. In some embodiments, the graphene is defective due to extrinsic defects resulting from perturbations in the crystalline order resulting from non-carbon atoms in the graphene. In some embodiments, the graphene is defective due to a combination of intrinsic and extrinsic defects. In some embodiments, the graphene is defective due to the presence of chemically doped nano regions comprising one or more of other atomic species. In some embodiments, the defects in the graphene comprise circular-shaped defects.

In some embodiments, the defects in the graphene comprise square-shaped defects. In some embodiments, the defects in the graphene comprise rectangle-shaped defects. In some embodiments, the defects in the graphene comprise irregular-shaped defects.

In some embodiments, the defects in graphene are disturbed lattice defects with a defect density between 10²/cm² and 10¹⁵/cm², for example, between 10⁴/cm² and 10¹⁰/cm² or between 10⁶/cm² and 10¹⁸/cm². In some embodiments, the defects in graphene are disturbed lattice defects with a defect density of 10⁵/cm²/ In some embodiments, the defects in the graphene are disturbed lattice defects with a defect density of at least 10⁵/cm². In some embodiments, the defects in the graphene are disturbed lattice defects with a defect density of 10⁵/cm², 10⁶/cm², 10⁷/cm², 10⁸/cm², 10⁹/cm², 10¹° /cm², 10¹¹/cm², 10¹²/cm², 10¹³/cm², 10¹⁴/cm², or 10¹⁵/cm².

In some embodiments, the defects in the graphene layer are nanoporous defects having a diameter of at least 0.2 nm with a defect density of at least 10³/cm². In some embodiments, the defects in the graphene layer are nanoporous defects having a diameter of between 0.5 nm and 20 nm, for example, between 0.5 nm and 15 nm, between 1 nm and 10 nm. In some embodiments the defect density is between 10³/cm² and 10¹⁵/cm², for example, between 10⁵/cm² and 10¹³/cm², or between 10⁷/cm² and 10¹¹/cm².

In some embodiments, the graphene is defective graphene, said defective graphene having a bandgap opened to a value of at least 0.2 eV or at least 0.5 eV. In some embodiments the defective graphene has a bandgap opened to a value of between 0.1 eV and 10 eV, for example, between 1 eV and 9 eV, between 2 eV and 8 eV, or between 3 eV and 7 eV, between 4 eV and 6 eV.

In some embodiments, the graphene is structured as a linear ribbon array with nanogaps between the graphene. In some embodiments, the width of each linearly parallel ribbon is between 1 and 500 nm, for example 1 nm, 10 nm, 20 nm, 30 nm, 40 nm, 50 nm, 60 nm, 70 nm, 80 nm, 90 nm, 100 nm 110 nm, 120 nm, 130 nm, 140 nm, 150 nm, 160 nm, 170 nm, 180 nm 190 nm, 200 nm, 210 nm, 220 nm, 230 nm, 240 nm, 250 nm, 260 nm 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm 440 nm, 450 nm, 460 nm, 470 nm, 480 nm, 490 nm, or 500 nm, or any value between the foregoing. In some embodiments, the width of each linearly parallel ribbon is less than 1 nm. In some embodiments, the width of any linearly parallel ribbon is greater than 500 nm. In some embodiments, the graphene structure is similar to the graphene structure depicted in FIG. 1(a).

In some embodiments, the graphene is a patterned-shaped ribbon array with gaps in the ribbon width between 2 and 500 nm. In some embodiments the gaps in the ribbon width is between 2 and 50 nm. In some embodiments, the graphene structure is similar to the graphene structure depicted in FIG. 1(b).

In some embodiments, the graphene structure displays periodic or random lattice defects. In some embodiments, the defects represent a vacancy type or interstitial type defect. In some embodiments, the gaps comprise 1-20 nm in size. In some embodiments, the gaps are less than 1 nm. In some embodiments, the gaps are greater than 20 nm. In some embodiments, the gaps are due to missing carbon atoms. In some embodiments, the gaps are due to missing phenyl rings. In some embodiments, the gaps are periodic. In some embodiments, the gaps are random. In some embodiments, the defect is due to a dopant effect. In some embodiments, the dopant effect is the result of attaching a nitrogen atom. In some embodiments, the dopant effect is the result of attaching a hydrogen atom. In some embodiments, the dopant effect is the result of attaching an atom that is neither nitrogen or hydrogen. In some embodiments, the dopant effect is due to the attachment of other functionalities. In some embodiments, the attachment occurs through chemical reaction means. In some embodiments, the attachment occurs through an ion implantation process. In some embodiments, the graphene structure is similar to the graphene structure depicted in FIG. 1(c).

In some embodiments, the defects introduced into the graphene are artificially introduced defects of a larger size, for example, between 2 and 100 nm. In some embodiments, the defects are periodic. In some embodiments, the defects are circular. In some embodiments, the defects are circular-shaped. In some embodiments, the defects are square-shaped. In some embodiments, the defects are rectangle-shaped. In some embodiments, the defects are irregular-shaped. In some embodiments, the graphene structure is similar to the graphene structure depicted in FIG. 1(d).

In some embodiments, the graphene layer is decorated with elongated nanowires selected from carbon nanotubes and semiconductor nanowires of doped Si, Ge, or ZnO. In some embodiments, the nanowire is a semiconducting nanowire. In some embodiments, the nanowire is a silicon nanowire.

In some embodiments, the graphene layer is positioned on each metallic electrode pair defining each nanogap such that the graphene layer is suspended over the substrate in each nanogap and not contacting the substrate in each nanogap. In some embodiments, the graphene layer does contact the substrate in each nanogap.

In some embodiments, the graphene layer is in contact with the dielectric substrate, with each pair of metallic conducting electrodes extending from both ends of the graphene layer as electrical lead wires. In some embodiments, the electrical lead wires comprises Au. In some embodiments, the lead wires are surface insulated or coated to prevent protein adhesion.

In some embodiments, the bonding between the polymerase enzyme molecule and the graphene layer comprises van der Waals interactions. In some embodiments, the bonding between the polymerase enzyme molecule and graphene layer comprises ionic bonding. In some embodiments, the bonding between the polymerase enzyme molecule and the graphene layer comprises covalent bonding. In some embodiments, the bonding between the polymerase enzyme molecule and the graphene layer comprises electrostatic forces.

In some embodiments, the bonding between the polymerase enzyme molecule and the graphene layer comprises a bifunctional linker bonded to the graphene. In some embodiments, the bonding between the polymerase enzyme molecule and the graphene layer comprises a bifunctional linker bonded at one end of the linker to the graphene via pi-stacking to a pyrene group.

In some embodiments, the bifunctional linker bonds to the polymerase enzyme molecule through functional binding pairs selected from streptavidin-biotin pair, mercaptocarbonic acid [HS-(CH₂)n-COOH, n=1-15] pairs, thiol-alkyne pair, COOH—NH₂ functional group pairs, thiol-maleimide pair, cysteine-maleimide pair, silanization linkage pairings using mercaptosilane compounds, an NHS (N-hydroxysuccinimide) ester-amine pair, an antigen-antibody pair, or a click chemistry pair. In some embodiments, the bifunctional linker bonds to the polymerase enzyme through covalent bonding. In some embodiments, the bifunctional linker bonds to the polymerase enzyme through ionic bonding. In some embodiments, the bifunctional linker bonds to the polymerase enzyme through van der Waals interactions.

In some embodiments, any of the sequencing structures described herein further comprise a gate electrode placed parallel to each source and drain electrodes or placed perpendicular to the nanogap spacing between each source and drain electrodes.

In some embodiments, the metallic conducting electrodes, graphene layer, dielectric masking layer, and enzyme polymerase molecule are arranged into an array configuration so as to allow massively parallel electronic sequencing analysis using devices organized into a system having at least 1,000 or at least 1 million devices. In some embodiments, the system has greater than 1 million devices, for example, greater than 2 million devices, greater than 3 million devices, greater than 4 million devices, greater than 5 million devices, greater than 6 million devices, greater than 7 million devices, greater than 8 million devices, greater than 9 million devices, or greater than 10 million devices.

In some embodiments, a sequential interrogation of an array of electrodes in the device is possible for DNA or genome sequencing by using a common lead wire on one side of the array. In some embodiments, the electrodes on the opposite side of the lead wire are connected to the common lead wire. In some embodiments, the electrodes on the opposite of the lead wire are sequentially interrogated one at a time. In some embodiments, the lead wire comprises Au. In some embodiments, the lead wires are surface insulated or coated to prevent protein adhesion.

In some embodiments, the metallic conducting electrodes, graphene layer, dielectric masking layer, and polymerase enzyme molecule are arranged into a three-dimensional array. In some embodiments, the metallic conducting electrodes, graphene layer, dielectric masking layer, and polymerase enzyme molecule are arranged into a three-dimensional array using a stacked microfluidic chamber or a common microfluidic chamber for a stacked layer of devices arrays, so as to allow massively parallel electronic sequencing analysis using devices organized into a system having at least 1,000, or at least 1 million devices, or at least 1 billion devices. In some embodiments, the system has greater than 1 million devices, for example, greater than 2 million devices, greater than 3 million devices, greater than 4 million devices, greater than 5 million devices, greater than 6 million devices, greater than 7 million devices, greater than 8 million devices, greater than 9 million devices, or greater than 10 million devices.

### Methods

In some embodiments, a method of fabricating a DNA sequencing device structure, said method comprising: (a) depositing and patterning an array of metallic conducting electrode pairs on a dielectric substrate, each pair defining a source and drain arrangement separated by a nanogap; (b) depositing a graphene layer over each metallic conducting electrode pair by using a liquid container lift-up placement method onto the array of metallic conducting electrode pairs, by using a vacuum transfer method, or by using a stamp transfer method, and processing the graphene to obtain a defective graphene material that bridges each source and drain in a pair while leaving each of the electrode pairs in electrical isolation from each other; (c) nanopatterning a dielectric masking layer on the defective graphene material, said masking layer having an opening exposing a portion of said defective graphene material directly over each nanogap, each opening dimensioned in size to match the size of a single polymerase enzyme molecule; (d) placing the construct resulting from steps (a) through (c) into a microfluidic system; and (e) supplying a solution of polymerase enzyme molecules to the microfluidic system to bond a single polymerase enzyme on the exposed portions of said defective graphene material, in the arrangement of one enzyme molecule per opening.

In some embodiments, the graphene layer is made defective by nanopatterning into linear nano-ribbon parallel array or patterned shape nano-ribbon array, or by introducing disturbed lattice defects, or by providing nanoporous defects. In some embodiments, the graphene is made defective by introducing intrinsic defects resulting from non-sp² orbital hybrid carbon atoms in graphene. In some embodiments, the intrinsic defect is a Stone-Wales defect. In some embodiments, the intrinsic defect is a single vacancy defect. In some embodiments, the intrinsic defect is a multiple vacancy defect. In some embodiments, the intrinsic defect is a line defect. In some embodiments, the graphene is made defective by introducing extrinsic defects resulting from perturbations in the crystalline order resulting from non-carbon atoms in graphene. In some embodiments, the graphene is made defective by introducing a combination of intrinsic and extrinsic defects.

In some embodiments, the graphene disturbed lattice defects are introduced by beam irradiation selected from ion implantation beam, plasma reactive ion etch (RIE) atmosphere, broadened or scanning optical, electron, ion or neutron beam, so as to introduce a defect density of at least 10⁵/cm². In some embodiments the defect density introduced is greater than 10⁵/cm², for example, greater than 10⁶/cm², greater than 10⁷/cm², greater than 10⁸/cm², greater than 10⁹/cm², greater than 10¹° /cm², greater than 10¹¹/cm², greater than 10¹²/cm², greater than 10¹³/cm², greater than 10¹⁴/cm², or greater than 10¹⁵/cm².

In some embodiments, the irradiated structure is post-irradiation annealed at 100-600° C. For example, the irradiated structure is post-irradiation annealed at 100° C., 110° C., 120° C., 130° C., 140° C., 150° C., 160° C., 170° C., 18° C. 0, 190° C., 200° C., 210° C., 220° C., 230° C., 240° C., 250° C., 260° C., 270° C., 280° C., 290° C., 300° C., 310° C., 320° C., 330° C., 340° C., 350° C., 360° C., 370° C., 380° C., 390° C., 400° C., 410° C., 420° C., 430° C., 440° C., 450° C., 460° C., 470° C., 480° C., 490° C., 500° C., 510° C., 520° C., 530° C., 540° C., 550° C., 560° C., 570° C., 580° C., 590° C., 600° C., or any temperatures between the foregoing. In some embodiments, the irradiated structure is post-irradiation annealed at less than 100° C. In some embodiments, the irradiated structure is post-irridiation annealed at greater than 600° C.

In some embodiments, the nanoporous defects are introduced by first depositing a thin film of metallic, ceramic or polymeric mask layer having 0.5-5 nm thickness on the surface of graphene layer by sputtering or evaporation method, followed by a beam irradiation selected from ion beam, electron beam, neutron beam, laser beam with optional post-irradiation annealing so as to produce a nanoscale mask having many pores, with an average pore diameter of 0.2-10 nm, with the graphene subsequently etched through the mask pores by oxygen plasma type treatment to introduce defects in the graphene with a density of at least 10¹⁰/cm.

In some embodiments, the graphene disturbed lattice defects or nanopores are introduced by chemical etching using oxidizing chemicals, strong acids, strong alkaline solutions, or potassium chlorate-containing solution so as to introduce a defect density of at least 10⁵/cm². In some embodiments, the defect density is greater than 10⁵/cm², for example, greater than 10⁶/cm², greater than 10⁷/cm², greater than 10⁸/cm², greater than 10⁹/cm², or greater than 10¹⁰/cm²

In some embodiments, the nanoporous defects are introduced by using block copolymer templated hole generation so as to introduce a defect density of at least 10⁵/cm². In some embodiments, the defect density is greater than 10⁵/cm², for example, greater than 10⁶/cm², greater than 10⁷/cm², greater than 10⁸/cm², greater than 10⁹/cm², or greater than 10¹/cm²

In some embodiments, the nanoporous defects are introduced by using anodized aluminum oxide templated hole generation so as to introduce a defect density of at least 10⁵/cm². In some embodiments, the defect density is greater than 10⁵/cm², for example, greater than 10⁶/cm², greater than 10⁷/cm², greater than 10⁸/cm², greater than 10⁹/cm², or greater than 10¹° /cm²

In some embodiments, the nanoporous defects are introduced by using nanoimprinting patterning so as to introduce a defect density of at least 10⁵/cm². In some embodiments, the defect density is greater than 10⁵/cm², for example, greater than 10⁶/cm², greater than 10⁷/cm², greater than 10⁸/cm², greater than 10⁹/cm², or greater than 10¹⁰/cm².

In some embodiments, the nanoporous defects are introduced by using nanoparticles spray deposited onto the graphene and performing chemical etching or oxidation reactions.

In some embodiments, a method is provided of maintaining the sequencing device of any of the sequencing devices described herein when the device is not in use comprising evacuating and back-filling the device with inert gas so as to minimize inadvertent adhesion or adsorption of unwanted molecules in the device.

In some embodiments, any of the sequencing devices described herein can be used to perform a whole genome sequencing,

In some embodiments, any of the sequencing devices describe herein can be used to diagnose diseases with a genetic component. In some embodiments, any of the sequencing devices described herein can be used to diagnose diseases with a genetic component, such as monogenic or polygeneic heritable diseases, somatic mutation disease such as cancer or tissue damage, infectious disease, such as viral or bacterial infection, or microbiome diseases or disorders.

In some embodiments, the system of any of the sequencing devices described herein is a desktop unit, a portable unit, a hand-held unit or a wearable unit.

In some embodiments, the electrode structures and supporting measurement circuity of any of the sequencing devices described herein are all embodied in pixels of a sensor array, which in turn is embodied in a semiconductor chip made via CMOS processing.

In some embodiments, a method of detecting and identifying an analyte, said method comprising: (a) depositing and patterning an array of metallic conducting electrode pairs on a dielectric substrate, each pair defining a source and drain arrangement separated by a nanogap; (b) depositing a graphene layer over each metallic conducting electrode pair by using a liquid container lift-up placement method onto the array of metallic conducting electrode pairs, by using a vacuum transfer method, or by using a stamp transfer method, and processing the graphene to obtain a defective graphene material that bridges each source and drain in a pair while leaving each of the electrode pairs in electrical isolation from each other; (c) nanopatterning a dielectric masking layer on the defective graphene material, said masking layer having an opening exposing a portion of said defective graphene material directly over each nanogap, each opening dimensioned in size to match the size of a single polymerase enzyme molecule; (d) placing the construct resulting from steps (a) through (c) into a microfluidic system and supplying a solution of polymerase enzyme molecules into the microfluidic system to bond a single polymerase enzyme onto each exposed portion of said defective graphene material, in the configuration of one molecule per opening; (e) supplying a fluid or series of fluids comprising at least one analyte requiring detection; and (f) making an electronic measurement and computer analysis of individual analyte- polymerase molecule interactions and/or incorporations as discrete events, to obtain electrical signals that determine the analyte identity.

In some embodiments, at least one analyte comprises DNA template molecules and substrates for nucleotide incorporation. In some embodiments, at least one analyte comprises RNA template molecules and substrates for nucleotide incorporation.

In some embodiments, the analyte identity comprises specific nucleotide base identity and a sequence of nucleotide incorporations by said polymerase enzyme molecules. In some embodiments, a method of producing graphene is provided, comprising: (i) using a carbon-containing gas as the precursor with Ni or Cu used as the metallic substrate, (ii) applying chemical vapor deposition, and (iii) etching after the graphene is synthesized. In some embodiments, the method of producing graphene produces a single-layer graphene. In some embodiments, the method of producing produces a few-layer graphene. In some embodiments, the few-layer graphene is between 2 and 5 layers. In some embodiments, the few-layer graphene is more than 5 layers.

In some embodiments, graphene is synthesized such that there is a bandgap in the graphene. In some embodiments, the bandgap is produced by intentionally disturbing the structure of graphene. In some embodiments, intentionally disturbing the structure of graphene produces a nanoribbon array. In some embodiments, intentionally disturbing the structure produces a nanomesh structure. In some embodiments, doping with other chemical species opens up the bandgap. In some embodiments, the bandgap value is between 0.2 and 4 eV. In some embodiments, the bandgap value is less than 0.2 eV. In some embodiments, the bandgap value is greater than 4 eV.

Referring to the drawings, FIG. 1 schematically illustrates exemplary defects that may be artificially introduced to a graphene sheet using various techniques to provide an increased bandgap in the resulting material, and also to provide active edge site locations for strong adhesion of additional bridge structures or biomolecules (such as an enzyme molecule). The graphene-derived material can be of armchair type, zig-zag type or chiral type.

Graphene is one-atom-thick or few-atoms-thick, two-dimensional, sp2 covalently bonded layer of carbon, with the carbon atoms organized in a hexagonal lattice arrangement with a very large lateral extent, ideally unbounded in all lateral directions, so that edge-effects are not significant. By virtue of its remarkable physical and chemical properties, graphene received much attention in recent years, with potential applications for advanced electronics, sensors, biotech, and energy technology.

Graphene can be obtained by cleaving of graphite such as by using a scotch tape, but a more common and reproducible fabrication technique is the well-established CVD (chemical vapor deposition) method using a carbon-containing gas as the precursor with Ni or Cu type metallic substrate which is chemically etched after the CVD synthesis. A single-layer graphene exhibits a well-defined structure and physical or chemical properties, but few-layer graphenes (e.g., a structure having 2-5 single graphene layers stacked) are also useful for some applications. When the number of layers is more than about 10, the material is no longer a graphene but is considered a graphite, lacking many of the unique properties of graphene.

Graphene is known to be a zero bandgap semiconductor material, such that there is no barrier to electrical conduction. To be useful for a semiconductor device, an energy bandgap needs to be established or “opened.” In various embodiments, a pristine graphene or a bandgap-opened graphene are used to derive material components for a DNA sequencing sensor system. In some embodiments, a bandgap can be opened by introducing intentionally disturbed structures such as subdivisions (e.g., into nanoribbon arrays or nanomesh structures) or nanoporous structuring with lattice structure extending nearly to the newly created edges. In some embodiments, a bandgap in graphene can be opened by chemical doping with metallic or nonmetallic elements. Creating vacancies or doping with interstitials can also open the bandgap. In some embodiments, the desired bandgap values of the graphene utilized for molecular sensors and for sequencing is in the range of 0.2-4 eV. In some embodiments, the desired bandgap is in the range of 0.5-3 eV. In some embodiments, the defective graphene also allows easier and stronger bonding with biomolecules defects on graphene edges provide many active sites for atomic bonding and chemical bonding.

Still referring to FIG. 1, the schematic drawing in FIG. 1a describes a linear ribbon array (1) with nanogaps (2) between adjacent graphene ribbons (with the graphene ribbon edges having arm-chair type, zig-zag type, or chiral type structures). The width of each of the linearly parallel ribbons may be in the range of 1-500 nm, such as for example, 2-50 nm. The desired gap spacing between adjacent ribbons may be in the range of 2-20 nm.

The schematic drawing in FIG. 1b describes a patterned-shape ribbon array (3) with gaps, with the ribbon width in the range of 2-500 nm, preferably 2-50 nm. The drawing of

FIG. 1c illustrates an example of periodic or random lattice defects in graphene (4). These defects represent a vacancy type or interstitial type defect, having a 1-20 nm size regime (e.g. from missing carbon atom(s) or phenyl rings, periodic or random), dislocation defect type, (e.g. as 5-7-7-5 pentagon-heptagon defects), or dopant defect type like nitrogen or hydrogen atoms and other atoms, and other functionalities attached, using chemical reaction means, ion implantation process, etc. Other compositionally doped regions can also be produced by incorporating one or more other atomic species such as nitrogen, sulfur, or metallic elements such as chemisorbed or adsorbed Co, Ni, Pd, Mn, Fe, Al, Ag, Cu, Au, and Pt. Such chemical doping is accomplished either during the synthesis of graphene by modifying the precursor for chemical vapor deposition, or by modifying the synthesized graphene layer by attaching dopant atoms or aggregates via diffusional reactions, chemisorption reactions or physical adsorbing reactions. The schematic in FIG. 1d describes an example of generally larger size, artificially introduced defects (periodic or random, e.g., 2-200 nm size) having circular, square, rectangle or irregular-shaped defects (5). These defects illustrated are somewhat larger than the dimension of lattice defects, and can be introduced by lithographic patterning (e.g., e-beam lithography, nano-imprint lithography, template-mask induced removal, such as using nanopinhole-containing (nanoporous) metallic, ceramic, or polymeric film mask on graphene, pre-patterned diblock co-polymer membrane, anodized aluminum oxide membrane followed by plasma (or RIE) etching to remove graphene material), or by irradiation with e-beam or ion beam.

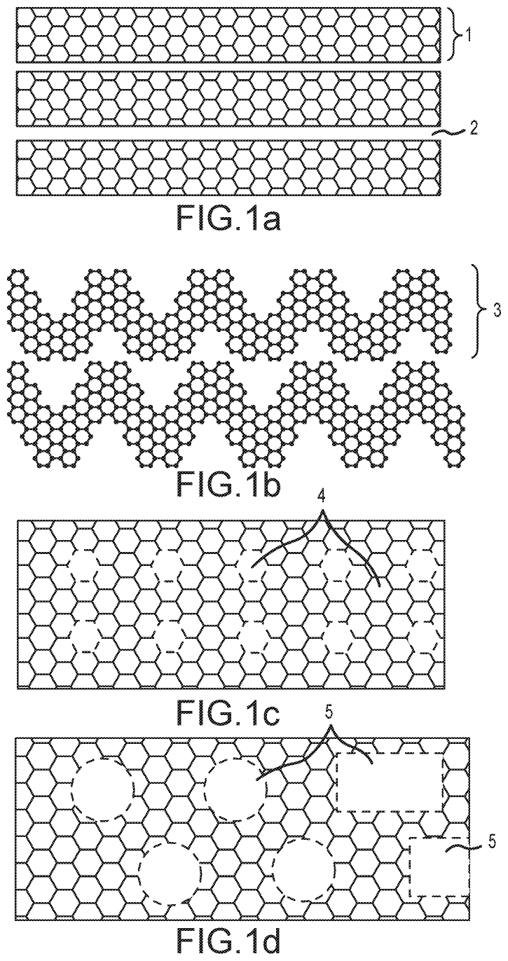

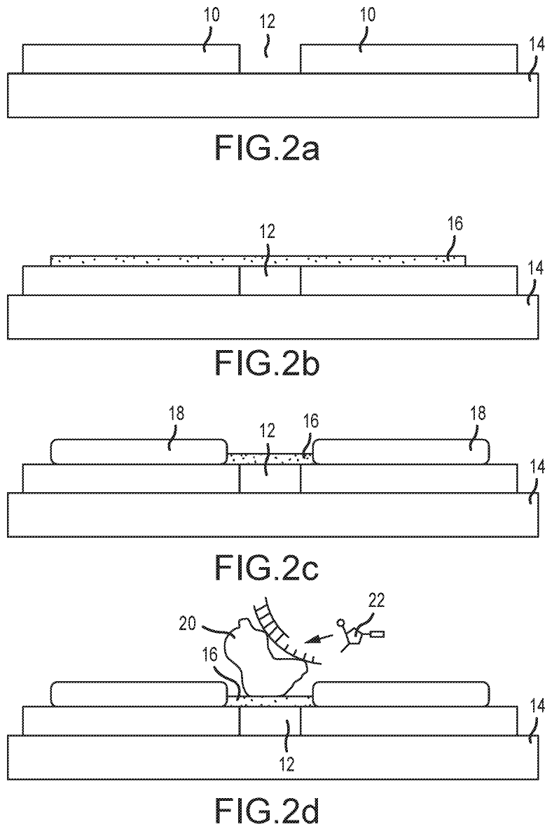

Referring again to the drawings, FIGS. 2a-d represent an embodiment of fabrication steps used to produce an exemplary molecular bridge of DNA or RNA sequencing sensor comprising a size-limiting, hanging (suspended) graphene-derived island region. FIG. 2a illustrates the first step wherein a conducting electrode pair (10) (e.g., made of highly conductive and corrosion resistant metals such as Au, Pt, Ag, Pd, Rh, or their alloys, etc. for signal conduction), with a nanogap (12) of e.g., 2-20 nm, is deposited and patterned on the surface of a dielectric substrate (14) like SiO₂, A1₂0₃, or a Si substrate with a relatively thick insulator layer of SiO₂, Al₂O₃, coated on the surface. In some embodiments, a nanogap of about 2-20 nm is provided between the two metal lead wires serving as a source and a drain. FIG. 2b illustrates addition of a suspended graphene layer (16) (regular graphene or a graphene structure intentionally made defective) over the electrode gap (12). One exemplary technique is to float the graphene layer on the surface of water or alcohol, and then lift up the substrate (with a pre-patterned metal electrode pair thereon) from underneath the water or alcohol to collect the graphene, followed by a drying procedure.

In some embodiments, the graphene sheet (16) is then coated with a protective dielectric coating (18) (e.g., polymer layer like polymethyl methacrylate (PMMA) or ceramic layer like SiO₂) and patterned, as illustrated in FIG. 2c, to limit the exposed area of the graphene to a nanometer sized regime island geometry (e.g., 2-20 nm average size). Specifically, FIG. 2c shows a protective size-limiting dielectric coating (18) (e.g., polymer layer like PMMA or ceramic layer like SiO₂). The graphene bridge is now size-limited (e.g. 2-20 nm) for preferably a single biomolecule (e.g., polymerase enzyme) attachment. Such a dimensional limitation (i.e. “size-limiting”) allows the preference of only a single molecule of enzyme (20) (e.g. DNA, RNA, or polymerase) to attach onto a size-limited graphene bridge, as illustrated in FIG. 2d, which allows a nucleotide monomer (22) (e.g., A, T, C, G, U etc.) to be detected on polymerase reaction. This dimensional limitation prevents multiple molecule attachment and the resulting signal mix-up. A single enzyme molecule (20) (e.g. DNA or RNA polymerase) also ensures that each enzyme-nucleotide interaction/attachment will result in a detectable change in the electrical properties of the molecular bridge. FIG. 2d also illustrates a molecular bridge solid state sensor on graphene for genome or DNA sequencing via detection of change of current pulse or other signals upon attaching or detaching of nucleotide or other biomolecules.

A polymerase is an enzyme that synthesizes long chains or polymers of nucleic acids. For example, DNA polymerase and RNA polymerase can copy a DNA or RNA template strand, respectively, by first binding to the template strand and then incorporating nucleotides from the free deoxynuclosidetriphosphate (dNTP) form in solution, guided by the well-known complementary base-pairing interactions (A-T, G-C, A-U). When a particular nucleotide or type of nucleotide or other biomolecule is interacting or engaged (attached) with the polymerase biomolecule (e.g. bound into the enzyme active site), a unique current signal is generated that provides information on what nucleotide or type of nucleotide molecule is being attached. Part of the sequencing operation includes the nucleotide incorporation complementary to the template strand, to form a double-stranded DNA (or DNA-RNA duplex) associated with polymerase.

Other non-limiting examples of biomolecules to be attached on the graphene-derived material, besides enzyme molecules discussed above, include various other polymers, DNA, RNA, proteins, antibodies, ribozymes, aptamers or polysaccharides. Other single molecule functionalizations of these defective graphene structures can provide sensors for other application areas besides genome sequencing. For example, an enzyme other than polymerase can be attached to produce a sensor for the activity of that particular enzyme. This can be used to sense the presence of the enzyme substrate, to characterize the precise kinetics of the enzyme, or to explore enzyme evolution, selection and optimization. If a single molecule binding probe is attached to the defective graphene, such as a single stranded DNA or RNA oligomer hybridization probe, or an antibody against an antigen, or a protein that engages in a protein-interaction binding, the resulting sensor can be used to sense the binding events, and thus acts as a sensor for the presence of the binding target.

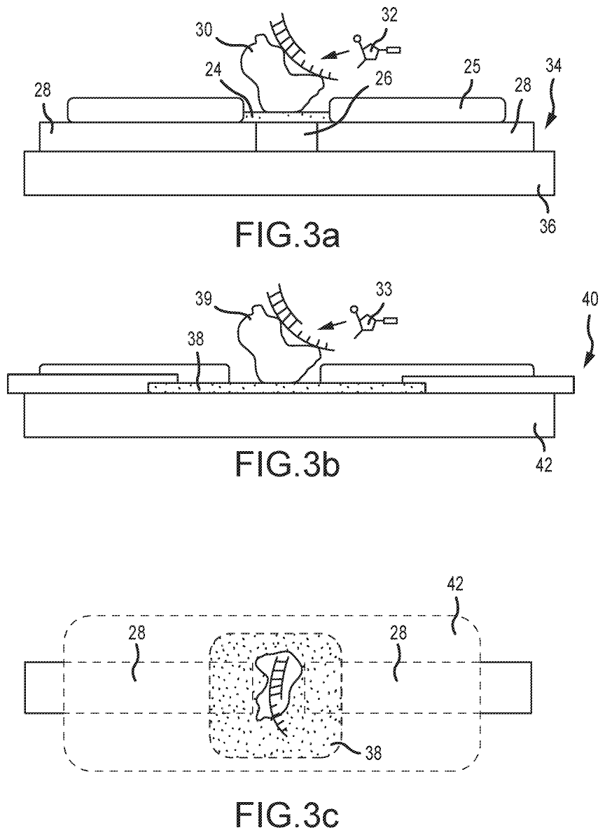

With reference now to FIGS. 3a-c, exemplary embodiments of two configurations of graphene bridge placement are illustrated. The schematic in FIG. 3a describes a suspended graphene bridge (24) (attached/affixed by Van der Waals force and dielectric coating), in which the graphene bridge (24) is suspended over a nanogap (26) between the electrode pair (28). As shown in FIG. 3a, a single enzyme molecule (30) (e.g., DNA or RNA polymerase) is attached onto a size limiting graphene bridge (24). Nucleotide monomers (32) (A, T, C, G, U, etc.) can be detected on the double stranded DNA. FIG. 3a also illustrates a molecular bridge solid state sensor (34) for genome or DNA sequencing detection via change of current or other signals upon attaching or detaching of biomolecules to polymerase, and incorporation of nucleotides. The dielectric substrate (36) is SiO₂, Al₂O₃ etc.

FIG. 3b shows a substrate-adhered graphene bridge (38). FIG. 3b illustrates a molecular bridge sensor (40) with the graphene layer (38) affixed onto the dielectric substrate (42), e.g. via van der Waals force and dielectric coating above. A top view of the graphene bridge structure with an attached polymerase enzyme is shown in FIG. 3c. As shown in FIG. 3c, graphene (38) (preferably defective or porous graphene) sheet is masked by dielectric substrate (42) (e.g., PMMA or adhesion-impeding polymer layer) to prevent biomolecule attachment. Conducting electrodes and lead wires (28) (Au, Pt, Ag, Pd, Rh, or other alloys, etc.) are used for signal detection. FIG. 3c further illustrates a size-limited, locally exposed graphene region (circular, square, or other shape) for single molecule attachment (e.g., 2-20 nm size).

As discussed herein, having only a single enzyme molecule as illustrated (as opposed to more than one enzyme on the graphene) enables each of the polymerase reactions involving nucleotide attachment (e.g., monomers like A, T, C, G, U, etc.) to change the electrical properties of the molecular bridge for sequencing analysis.

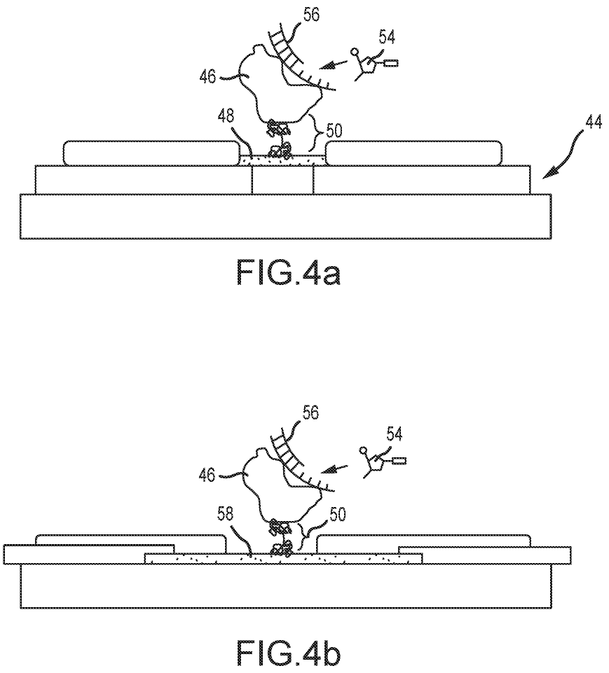

The defective graphene, also allows an easy and strong bonding with biomolecules as the graphene edges and defects provide many active sites for atomic bonding and chemical bonding. While a direct adhesion of biomolecules, such as a polymerase enzyme molecule, is more desirable for simpler structuring and less interfacial electrical resistance, the embodiments disclosed herein do not exclude use of a functional linker to ensure stronger attachment of biomolecules onto graphene (as discussed herein below in the context of FIG. 4). In various embodiments, the linker may be covalently bonded to both the graphene layer and the enzyme (or other biomolecule). In other embodiments, the bonding of the linker on either end may comprise any combination of bonding types (covalent, ionic, Van der Waals, etc.).

FIGS. 4a-b illustrates exemplary bonding of an enzyme molecule to the graphene via the aid of functional groups, (i.e. a bifunctional linker, also called a tether). Two exemplary embodiments of a solid state molecular sensor are illustrated: FIG. 4a details a suspended graphene bridge, and FIG. 4b details a substrate-adhered graphene bridge. FIG. 4a illustrates a molecule bridge solid state sensor (44) for genome or DNA or RNA sequencing detection via change of current or other signals upon attaching or detaching of biomolecules or nucleotide incorporation. The conducting electrodes and lead wires (Au, Pt, Ag, Pd, Rh, or their alloys, etc.) are used for signal detection. The bottom layer is the dielectric substrate (SiO₂, Al₂O₃, etc.). A nanogap is present between the electrode pairs. As shown in FIG. 4a, an enzyme molecule (46) (e.g. DNA or RNA polymerase) is linked to a suspended graphene bridge (48) using a linker (50). Functionalizing groups on the linker, link the enzyme molecule to the suspended graphene bridge (48). Nucleotide monomers (54) (A, T, C, G, U etc.) are detected on the double stranded DNA (56). FIG. 4b illustrates a molecular bridge sensor with the graphene layer affixed onto the dielectric substrate via Van der Waals force and dielectric coating above. As shown in FIG. 4b, an enzyme molecule (46) (e.g. DNA or RNA polymerase) is linked to a substrate-adhered graphene bridge (58) using a linker (50). Functionalization groups on the linker link the enzyme molecule to the graphene. Nucleotide monomers (54) (A, T, C, G, U etc.) are detected on the double stranded DNA (56). In some embodiments, a defective or nanoporous graphene layer is utilized, which is masked to a limited size area (e.g., 2-20 nm) to promote only single molecule attachment. Masking may be accomplished by using a patterned dielectric coating (e.g., PMMA, SU8/polydimethylsiloxane (PDMS) or other adhesion-impeding polymer layer, or SiO₂ layer). The enzyme molecule may attach to the surface of the graphene by non-specific adhesion (e.g., van der Waals or other interaction), or through a bifunctional linker to bond the enzyme molecule to the material surface. The carbon surface can be bound to via pi-stacking to a pyrene group or other group that is adherent to graphene or that engages in pi-stacking bonds. The other functional group (i.e. the other end of the linker) should itself engage in a pairing (a conjugation) that can support enzyme binding. Such other functionalization paired groups that can be used as conjugation pairs for molecular conjugation to link the enzyme or other biomolecule (which may itself be modified or preconjugated to present the suitable partner group for pairing) to the graphene include, but are not limited to, a “click chemistry” pair, an avidin-biotin pair, bifunctional ligands using mercaptocarbonic acids [HS-(CH₂)n-COOH, n=1−15], peptide functional groups, antibody-antigen pair, thiol-alkyne pair, COOH—NH2 functional group pair, a thiol-maleimide pair, a cysteine-maleimide pair, silanization linkage using mercaptosilane compounds, and a NHS (N-hydroxysuccinimide) ester-amine pair.

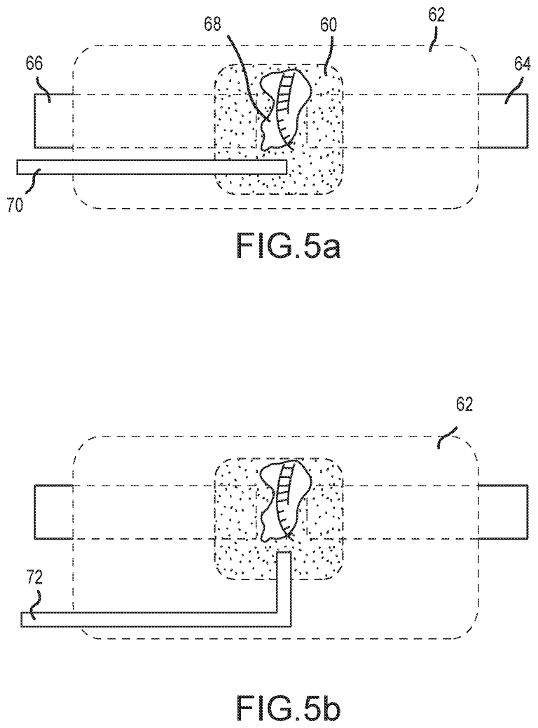

The graphene-containing molecular bridge structure for sequencing can comprise two electrodes (source and drain), or may optionally comprise a third electrode functioning as a gate electrode, as illustrated in FIGS. 5a-b, which shows a top view of graphene bridge single molecule sensor with a gated structure. Gated structure of a graphene bridge sequencing device can have the gate electrode positioned parallel to the source and drain electrodes (as shown in FIG. 5a) or perpendicular to the nanogap spacing between the two (source and drain) electrodes (as shown in FIG. 5b). FIG. 5a illustrates a graphene (preferably defective or porous graphene) sheet (60), masked by dielectric (62) to prevent biomolecule attachment. The dielectric masks the graphene except for a selected limited area. The conducting electrodes (source & drain) (64, 66, respectively) contain a nanogap (68) (e.g., 2-50 nm). There is a gate electrode (70) facing the device junction in a parallel geometry for triode type sensor operation. FIG. 5b is similar to FIG. 5a except that the gate electrode (72) faces the device junction in a perpendicular way for triode type sensor operation. The tip of the gate electrode can also be placed above or below the nanogap, including the possibility that it is buried below the gap, inside the substrate, in a so-called backgate configuration. Buried gate configurations, such as with the gate structure buried below the substrate and possibly positioned directly under the gap region or limited area region, may be advantageous.

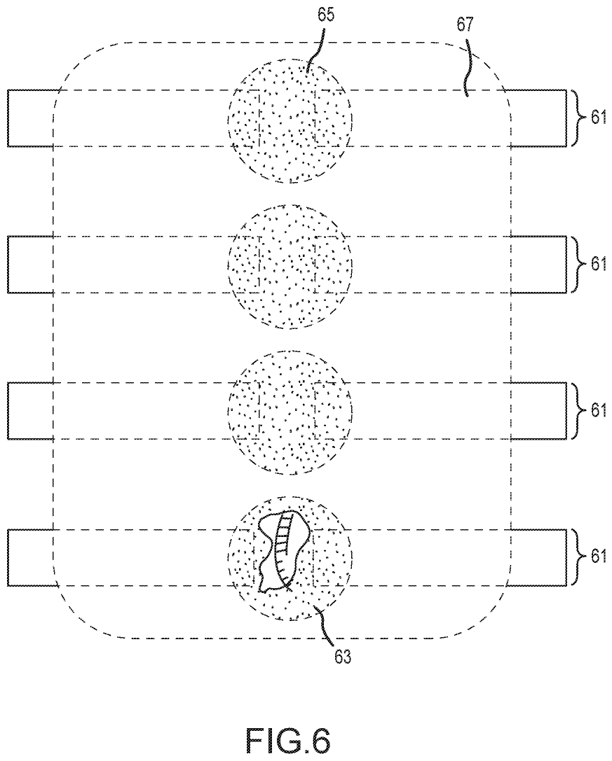

FIG. 6 illustrates a top view of an array of graphene bridge molecular sensors, with the graphene positioned as a suspended layer or substrate-adhered layer. FIG. 6 shows multiple units of molecular bridge sensors (61) comprising a graphene layer bridge. The polymerase enzyme molecule (63) is present on all four electrode pairs but is shown only on the lowest electrode pair in FIG. 6. FIG. 6 illustrates a size-limited, locally exposed graphene (65) (preferably defective or porous), with the exposed island region mask pattern defined electric coating (e.g., PMMA, PDMS, other adhesion-impeding polymer layer or SiO₂ etc.). FIG. 6 also illustrates multiple arrays of conducting electrode and lead wires (67) (Au, Pt, Ag, Pd, Rh, or their alloys, etc.) for signal detection. A multitude of electrode pairs (e.g., made of Au, Pt, Ag, Pd, Rh, or their alloys) for electrical signal measurements can be constructed as shown in FIGS. 2 through 6, using e-beam lithography, nanoimprint lithography, and other known nanofabrication methods. A graphene layer (including in embodiments in which the graphene layer is defective or nanoporous) is deposited on the electrode pair array, e.g., by using a lift-up process from aqueous or alcohol bath with a floating graphene sheet, thus covering hundreds or thousands of electrode pairs as needed. The graphene sheet covering many electrode pairs is then patterned so as to isolate the graphene region for each electrode pair from the neighboring graphene regions.

The graphene sheet is then coated with a protective dielectric coating (e.g., polymer layer like PMMA or ceramic layer like SiO₂) and patterned, to limit the exposed area of the graphene to nanometer regime island geometry (e.g., 2-20 nm average size). The dimensional limitation allows preferably only a single enzyme molecule (e.g. polymerase) to attach onto each graphene island region as shown in FIG. 6. Massively parallel electronic sequencing analysis can be performed with many devices organized into a system, having e.g., as many as 10,000 or 1 million or 10 million or more devices. The single enzyme molecule (e.g. polymerase) on each of the graphene islands is such that the polymerase reaction of nucleotide incorporation (nucleotide monomers like A, T, C, G, U, etc., incorporated from their in-solution form of deoxynuclosidetriphophates (dNTPs)) changes electrical properties of the molecular bridge and thereby provides a signal for sequencing analysis.

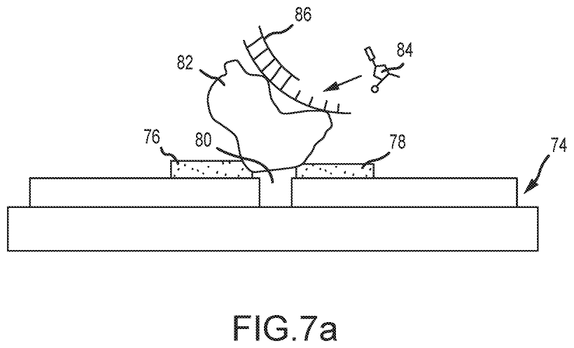

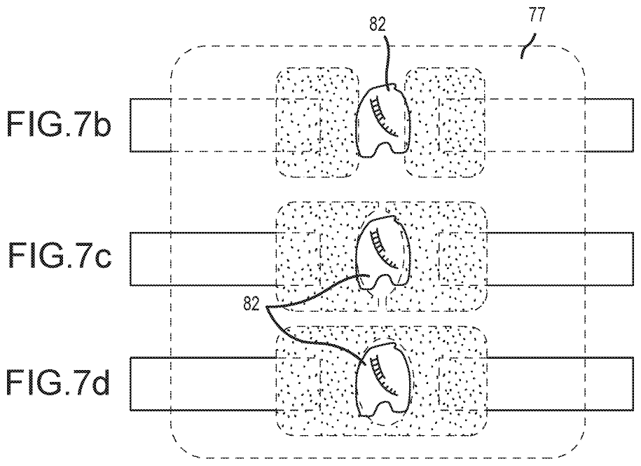

Shown in FIGS. 7a through 7d are embodiments of a molecular bridge solid state sensor (74) comprising a nanogapped graphene derivative for genome or DNA sequencing detection. The graphene island is split into two regions (76, 78) with a nanogap (80) of 2-20 nm between the two graphene regions. There is a dielectric mask layer (e.g., PMMA, PDMS, other adhesion-impeding layer or SiO₂ etc.) to prevent biomolecule attachment. When a nucleotide or other molecule is interacting with the enzyme biomolecule, a unique current signal is generated at the split-graphene tunnel junction, which provides information on which nucleotide is being incorporated or what type of molecule is present and the nature of the interaction (such as, for example, DNA template binding). The drawing of FIG. 7a shows a side view of an embodiment of a molecular bridge solid state sensor comprising nanogapped graphene for genome or DNA sequencing detection. The molecular bridge solid state sensor for genome or DNA or RNA sequence detects change of current or other signals upon attaching or detaching of biomolecules or nucleotide incorporation. Conducting electrodes and lead wires (Au, Pt, Ag, Pd, Rh, or their alloys, etc.) are used for signal detection. Between the electrode pairs is a nanogap. The sensor uses a split, nanogapped graphene regions as a well-adhereable substrate for tunnel junction construction. FIG. 7a illustrates a single enzyme molecule (82) (e.g. DNA or RNA polymerase) attached on the size-limited graphene bridge. The single enzyme molecule attaches onto the graphene nanogap in a tunnel junction configuration. A nucleotide monomer (84) is attached to the double stranded DNA (86). Change of current or other signals are measured upon incorporation of nucleotides or other interactions of the enzyme with biomolecules at the split-graphene tunnel junction configuration (such as DNA template binding). The drawing of FIG. 7b shows a top view of a single enzyme molecule (82) seated and edge-adhered onto a nanogap graphene. The nano-gapped graphene configuration with the gap spacing intentionally made comparable to the width of a single enzyme molecule (e.g., a single DNA polymerase enzyme biomolecule) helps the enzyme biomolecule to be more firmly attached onto the active edges of the gapped graphene due to the close proximity of the active edges to the molecule. In the embodiment illustrated in FIG. 7c, the curved graphene edge shape in a nanogapped but round or oval hole shaped region (with removed graphene from the middle) allows the enzyme molecule (82) to be seated and well-adhered to the graphene edges. The presence of nanogap allows tunneling type signal extraction from the nucleotide incorporation events, or other interactions between the polymerase enzyme and biomolecules or the bridge structure. FIG. 7d illustrates graphene with an intentionally induced round or oval hole region to provide a region in which the enzyme molecule can seat and well-adhere to the edge of such a hole. In the embodiment illustrated in FIG. 7d, the benefit of the tunneling effect is not utilized but the stronger adhesion of the enzyme molecule is advantageous for the stability of the molecular bridge structure and an enhanced generation of electrical signals.

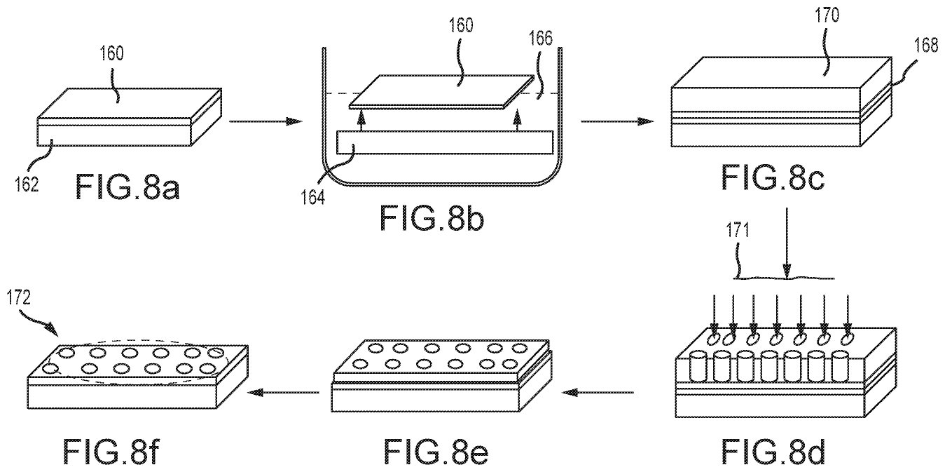

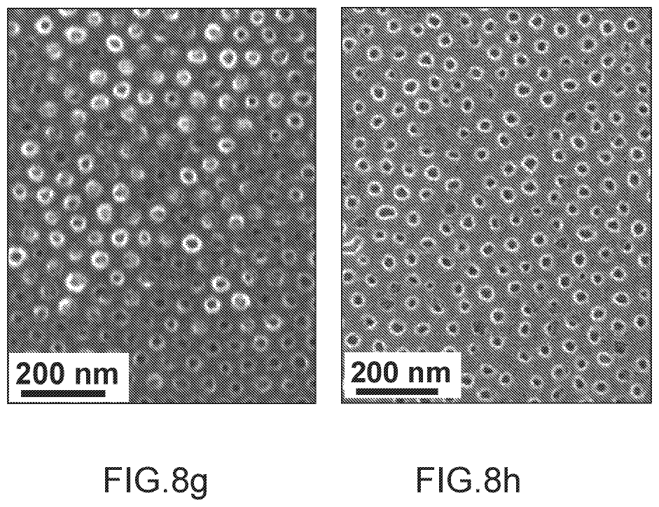

Graphene can be processed using nanotemplating to induce a very high density of nanopores, which can increase bandgap as well as enhance the adhesion of the biomolecule (e.g., a molecule of DNA or RNA polymerase) onto the graphene. Shown in FIGS. 8a-f is an exemplary fabrication procedure for a nanopatterned graphene bridge using diblock copolymer mask. In various embodiments of the present disclosure, fabrication steps for a nanopatterned graphene bridge using diblock copolymer mask may comprise: growing graphene on Cu foil using CVD to form the starting material (as shown in FIG. 8a); after etching the Cu with 1M FeCl₃, transferring the graphene on SiO₂ (or SiO₂-coated Si), or Au-electrode-pair deposited SiO₂ to collect floating graphene as a suspended bridge (the graphene can be suspended in H₂O or alcohol) (as shown in FIG. 8b); covering the graphene with a thin layer of evaporated SiO₂ and a thin film of spin-coated block-copolymer PS-b-P4VP (as shown in FIG. 8c); annealing and developing the PS-b-P4VP block-copolymer film into a nanoscale two-phase structure and etching away one of the phases, leaving the porous PS matrix (171) as the template for subsequent patterning (as shown in FIG. 8d); performing fluoride-based reactive ion etching (RIE) to penetrate and pattern the SiO₂ oxide layer, partially degrade the PS film, and form the hard mask hole pattern out of the thin SiO₂ (as shown in FIG. 8e); etching away the graphene in the exposed hole area by O₂ plasma and removing the SiO₂ (as shown in FIG. 8f). Finally, nanoporous graphene on SiO₂ was obtained, which is then patterned into a limited size island graphene by dielectric masking for single molecule (e.g., an enzyme) attachment.

The drawing of FIG. 8a illustrates a CVD grown graphene (160) on a Cu or Ni foil (162), forming the starting material. In FIG. 8b, after Cu substrate was etched away, e.g., by 1M FeCl₃, the graphene (160) was “lift-up transferred” (as discussed herein) onto SiO₂ (or SiO₂-coated Si) substrate (164), or onto an Au-electrode-pair fabricated on the substrate, so as to collect graphene as a suspended bridge. An alternative method of placing the graphene layer on the substrate is to utilize a transfer method, e.g., using a gentle vacuum device or a stamp to pick up the graphene sheet and release it at the desired location on the substrate surface, optionally using a releasing agent material at the interface between the pick-up device and the graphene layer.

In FIG. 8c, graphene was covered by a thin layer of evaporated SiO₂ (162) and a thin film of spin-coated block-copolymer PS-b-P4VP (170). FIG. 8d shows annealing and developing the PS-b-P4VP block-copolymer film into a nanoscale two-phase structure, leaving the porous PS matrix (171) as the template for subsequent patterning.

The graphene layer with diblock copolymer template on top is then etched by using fluoride-based reactive ion etching (RIE) to penetrate and pattern the SiO₂ oxide layer, partially degrading the PS film, and forming the hard mask hole pattern out of the thin SiO₂, as illustrated in FIG. 8e. Graphene in the exposed hole area was etched away by O₂ plasma, followed by SiO₂ removal. Finally, nanoporous graphene on SiO₂ (172) was obtained (as shown in FIG. 8f), which is then patterned into a limited size island graphene by dielectric masking for single molecule (e.g., enzyme) attachment.

Shown in FIGS. 8g and 8h are SEM micrographs depicting the process of nanopatterning of graphene using diblock copolymer mask. The micrograph of the phase decomposed block-copolymer film (hexagonally distributed P4VP domains in the PS matrix) on graphene is shown as FIG. 8g. The SEM image of FIG. 8h represents nanopatterned graphene surface after SiO₂ mask patterning, graphene removal from the mask hole regions, and etching away of SiO₂ mask. The nanopores are 30 nm diameter. Various types of diblock copolymers or triblock copolymers can decompose into different types and sizes of two phase structures, and the block copolymer masked patterning approach can produce ˜5-50 nm size defects on graphene for bandgap opening and enhanced biomolecule adhesion. The desired density of artificially introduced nanopores on graphene by block copolymer template approach is at least 10⁵/cm², preferably at least 10⁷/cm², more preferably at least 10¹⁰/cm², and most preferably at least 10¹²/cm².

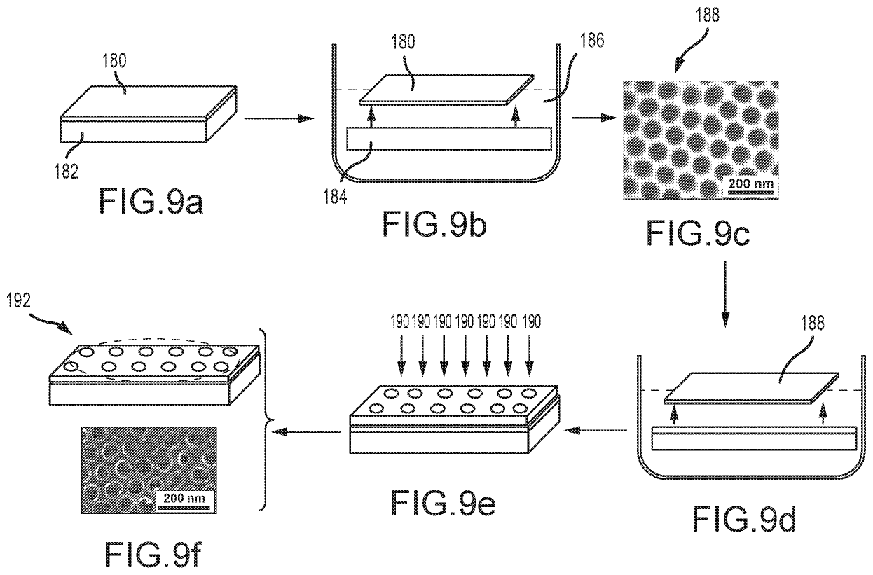

Other templates other than diblock copolymers can be utilized for nanoporous graphene synthesis, e.g., AAO (anodized aluminum oxide) membrane. Shown in FIGS. 9a-f is an AAO membrane based nanopatterning of graphene for molecular bridge sensors useful for DNA sequencing. The CVD grown graphene (180), shown in FIG. 9a, is placed on a substrate (184) by a lift-up process, as shown in FIG. 9b. The fabricated AAO nanohole membrane (188) (FIG. 9c) is placed on a graphene-coated substrate surface (as shown in FIG. 9d), followed by RIE etching of graphene through the AAO holes (FIG. 9e) and removal of AAO template to obtain nanoporous graphene (192) (as shown in FIG. 9f). The desired density of artificially introduced nanopores on graphene by AAO template approach is at least 10⁵/cm², preferably at least 10⁷/cm², more preferably at least 10¹⁰/cm², and most preferably at least 10¹²/cm².

More specifically, the drawing in FIG. 9a illustrates CVD grown graphene (180) on a Cu or Ni foil (182) forming the starting material. In FIG. 9b, the Cu substrate was etched away, the graphene was “lift-up transferred” onto SiO₂ (or SiO₂-coated Si) substrate (184) or onto a n Au-electrode pair fabricated on the substrate, so as to collected graphene as suspended bridge. FIG. 9c illustrates AAO membrane (188) synthesis (e.g. by anodization of AI foil) and dissolving away the AI matrix. FIG. 9d illustrates an AAO membrane in liquid such as H₂O or alcohol and a graphene coated substrate lifted up to pick up the AAO membrane. FIG. 9e shows reactive ion etching through AAO masked holes (190) after a dry AAO coating is added. FIG. 9f shows a nano patterned graphene (192) after removal of the AAO.

Such defective graphene layers obtained by nanopore generation as described herein may be additionally post-etch annealed (e.g., at 100°-600° C. for 10 minutes to 24 hrs) for further bandgap changes and easier biomolecule attachment.

Yet another embodiment to generate nanoporous graphene is to utilize nanoimprinting patterning so as to introduce a defect density of at least 10⁵/cm². A master nanoimprinting mold (stamp), e.g., with 10 nm regime nanoislands, nanoholes or nanolines can be generated by e-beam lithography. Many daughter stamps can then be generated using known pattern transfer techniques to allow nanopatterning of many devices.

It should be noted that the methods of making defective grapheme layers such as described in FIGS. 8-9 are for the purpose of demonstrating the principle that nano-defective graphene can be experimentally prepared by utilizing masks or templates. Variations or further optimized techniques of such inventive approaches can be employed, which is under the scope of the embodiments described herein. For example, by using smaller dimensioned masks and templates, finer dimensional scale defects in graphene such as 0.2-5 nm dimensions, can be produced, according to the embodiments described herein as described below.

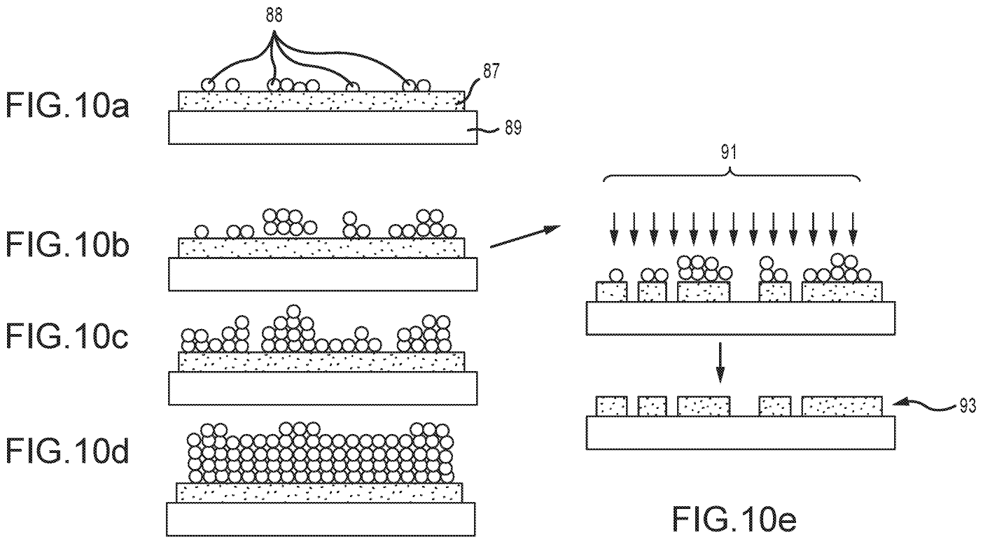

Referring back to the drawings, FIGS. 10a-e schematically describe the evolution of a thin film structure during deposition, e.g., by sputtering or evaporation. During the very early stage of deposition, as shown in FIG. 10a, atoms or molecules (88) are deposited in the form of individual atoms or islands, which then grow to be laterally connected (as shown in FIGS. 10b and 10c) to eventually form a desirably continuous layer configuration as shown, for example, in FIG. 10d.

For typical good thin film deposition, a pinhole-free structure as shown in FIG. 10d is desirable. However, in some embodiments, the desired structure is the opposite of such a good thin film mask material, so a shorter deposition time to achieve a thinner layer coating is desirable to obtain a pin-hole-containing mask layer of FIG. 10b or FIG. 10c.

In some embodiments, in order to obtain a high-quality thin films, typically the density of pinholes or vacancies is to be reduced as much as possible. In some embodiments, a processing and structurally approach opposite to this is desired, with the preferred density of such vacancy or pinhole defects at least 10⁵/cm² area, more preferably at least 10⁷/cm², even more preferably at least 10¹⁰/cm².

Also, to reduce the pinholes in sputter deposition for high quality thin films, enhanced atomic diffusion is often provided, such as a higher substrate temperature. In some embodiments, the opposite is desired, so the thin film mask deposition is carried out preferentially at lower temperature, e.g., <100° C., preferably near or below room temperature of ˜20° C., more preferably at low temperatures of e.g., −20° C. or lower.

In addition, in some embodiments, for the purpose of introducing more defects and pinholes, the mask film thickness is intentionally reduced to preferably less than 10 nm, more preferentially less than 5 nm. The mask material can be selected from metallic materials such as a thin Ni or Co, Mo, W, various metals and alloys, a ceramic or compound layer such as SiO₂ or NaCl, a sputter depositable polymer layer such as polytetrafluoroethylene (PTFE).

An optional post-sputtering heat treatment at temperatures, e.g., at or below ˜600° C. for less than 8 hrs, may be provided so as to consolidate the nanopinholes to a more uniform size distribution.

The next step, as shown in FIG. 10e, is to etch the graphene (91) through the mask defects, e.g., by using oxygen plasma etching of graphene material only where the pinholes are present.

Once the nanopinhole defects are prepared in the graphene, the mask material is removed as shown in FIG. 10e. To conveniently remove the film mask from the surface of grapheme, the metallic or ceramics film mask material can be etched away by a solution containing one or more acids such as HCl, HNO₃, H₂SO₄ or their mixtures. The ceramic mask such as SiO₂ can be etched away by diluted HF acid or a BOE (buffered oxide etch) solution such as 6:1 volume ratio of 40% NH₄F in water to 49% HF. The mask layer can also be etched away by plasma etching or ion beam etching. If NaCl or other salt type material is sputter deposited and employed as the mask layer, it can be dissolved away by water or solvents. Polymer mask films can be removed by solvent or by plasma etching.

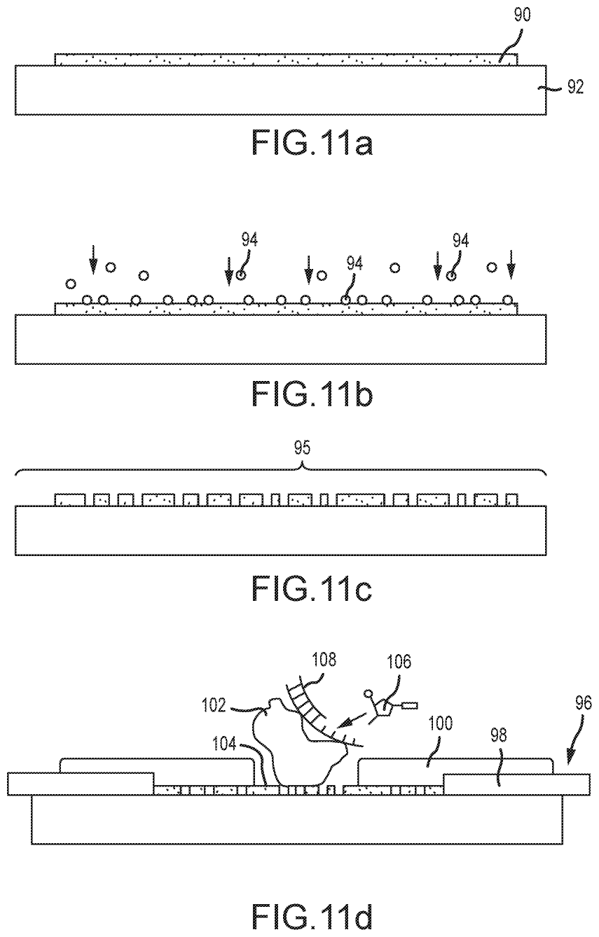

According to another embodiment, another method to generate perforated defective graphene is to utilize nanoparticles as illustrated in FIG. 11. In various embodiments, the method comprises: (a) providing a graphene layer; (b) coating the surface of the graphene layer with nanoparticles (NPs) comprising ceramic or metal; (c) inducing oxidation or degradation reactions by heating followed by etching removal of the reacted NPs to obtain perforated graphene; and (d) adding electrical lead wires and size-limiting dielectric layer (polymer or ceramic).

Referring to the drawings again, FIG. 11a illustrates a graphene layer (90) deposited on a dielectric substrate (92) (SiO₂, Al₂O₃, etc.). FIG. 11b illustrates nanoparticles (NP) (94) added on graphene such as sprayed, liquid-dispersion-coated and dried, or solid film deposition coated and balled-up by annealing (e.g., Ag, Ni, Cu islands). FIG. 11c illustrates perforated defective graphene (95) by reaction with 1-20 nm size nanoparticles (e.g., local graphene oxidation on heating by Fe₂O₂, CuO, other oxide NPs or Fe, Ni, Ti, Mo, V, Ta and other metallic NPs, especially carbide forming metals). Etching away of the reacted NPs induces perforated graphene for bandgap opening and easier biomolecule attach. Nanoparticles (NP) can be added on graphene e.g., by spraying, liquid-dispersion-coating and drying, or solid film deposition coating and balling-up by annealing (e.g., Ag, Ni, Cu islands). A similar approach is to produce a defective graphene oxide, then reduce to graphene. FIG. 11d illustrates a molecular bridge solid state sensor (96) for genome or DNA sequencing detection via a change of current or other signals upon attaching or detaching of biomolecules or nucleotide incorporation. The sensor comprises electrical lead wire (98) (Au, Pt, Pd, alloys) and dielectric material (100). A single enzyme molecule (102) (e.g. DNA or RNA polymerase) is attached onto a size-limited graphene bridge (104). Nucleotide monomers (106) (A, T, C, G, U etc.) are detected on the double stranded DNA (108).

An alternative technique of forming nanopores on graphene using nanoparticles is to have the NPs deposited first, then coat with Au or a polymer layer, remove the NPs, plasma etch the uncovered regions, and remove the Au or polymer protective coating. A similar approach to produce defective graphene oxide and then reduce to graphene is also an alternative embodiment.

The desired density of artificially introduced nanopores on graphene in accordance to the method illustrated in FIG. 11 is at least 10⁵/cm² preferably at least 10⁷/cm², even more preferably at least 10¹⁰/cm². On such nanoporous graphene, one can add electrical lead wires and size-limiting dielectric layer (polymer or ceramic) to make the solid state electronic sensor for single enzyme molecule and associated analytes attachment and sequencing analysis. The single enzyme (polymerase) molecule on graphene enables each of the polymerase reactions of nucleotide incorporation (nucleotide monomers like A, T, C, G, U, etc.) to uniquely change electrical properties of the molecular bridge for DNA or RNA sequencing analysis.

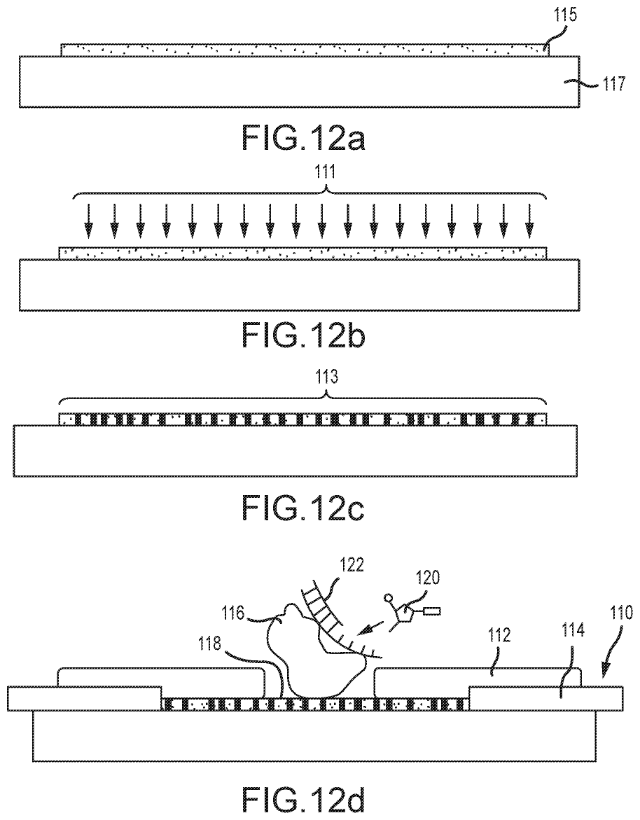

Instead of using a phase decomposition, a porous mask, nanoparticles, or various other approaches, a defective graphene can also be obtained by a beam irradiation for enhanced molecular bridge DNA or RNA sequencing sensors, as shown in FIG. 12. In various embodiments, the method exemplified in FIG. 12 comprises: providing a graphene layer (115) on a dielectric substrate (117) (as shown in FIG. 12a); irradiating the graphene with ion implantation beam (111) (e.g., with N, C, F, O, H, Ar, Ni, Ti, Mn, Fe, Cu, Ai and other species), plasma reactive ion etch (RIE) atmosphere (such as oxygen, argon or ammonia plasma), broadened or scannable optical, electron, ion or neutron beam to obtain induced defective graphene (113) (as shown in FIG. 12b); optionally performing a post-irradiation annealing (e.g. 100-600° C.) to induce point defects, interstitials, or agglomerated NP or nanopore defects for bandgap change and easier biomolecule attach (as shown in FIG. 12c); and adding electrical lead wires and size-limiting dielectric mask layer (polymer or ceramic) to make the solid state electronic sensor for single enzyme molecule and associated analyte attachment and sequencing analysis (as shown in FIG. 12d). Graphene can be irradiated, for example, by ion implantation beam, plasma reactive ion etch (RIE) atmosphere, broadened or scannable optical, electron, ion or neutron beam. The defective graphene thus obtained, with an optional addition of post-irradiation annealing, can be combined with electrical lead wires and size-limiting dielectric mask layer (polymer or ceramic) to make the solid state electronic sensor for single enzyme molecule attachment and subsequent sensing of DNA template attachment and nucleotide incorporation for sequencing analysis. The desired density of defects in the graphene is at least 10⁵/cm², preferably at least 10⁷/cm², and even more preferably at least 10¹⁰/cm². DNA, RNA or genome sequencing analysis is enabled when the single enzyme molecule (polymerase) on graphene causes each of the polymerase reactions of nucleotide incorporation (nucleotide monomers like A, T, C, G, U, etc.) to uniquely change electrical properties of the molecular bridge sensor.

FIG. 12d illustrates a molecular bridge solid state sensor (110) for genome or DNA sequencing detection via change of current or other signals upon attaching or detaching of biomolecules or nucleotide incorporation. The sensor comprises a size-limiting dielectric mask layer (112) and electric lead wires (114). A single enzyme molecule (116) (e.g. DNA or RNA polymerase) is attached onto a size-limited graphene bridge (118). Nucleotide monomers (120) (A, T, C, G, U etc.) are detected on the double stranded DNA (122).

Such defective graphene layers obtained by beam irradiation as described in FIG. 12 may be additionally post-etch annealed (e.g., at 100-600° C. for 10 minutes to 24 hrs) for bandgap change and easier biomolecule attachment.

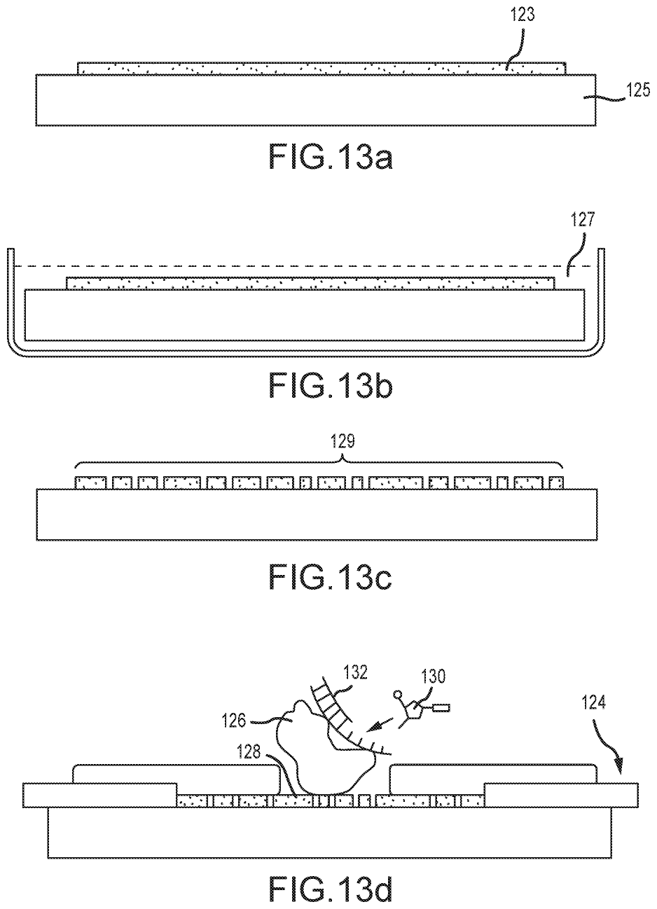

Shown in FIG. 13 is a chemically induced defective graphene (disturbed lattice regions or nanopores). In various embodiments, chemically induced defective graphene having disturbed lattice regions or nanopores may be obtained by a method comprising: providing a graphene layer (123) on a substrate (125) (as shown in FIG. 13a); chemically etching the surface of the graphene layer to obtain defective graphene (as shown in FIG. 13b); optionally performing a post-irradiation annealing (as shown in FIG. 13c); and adding electrical lead wires and size-limiting dielectric layer (polymer or ceramic) to make the solid state electronic sensor for single enzyme molecule and associated analyte attachment, incorporation and sequencing analysis (as shown in FIG. 13d). The graphene layer on a sequencing solid state sensor substrate (as shown in FIG. 13a) is immersed in a chemical etching or electrochemical etching bath (127) (as shown in FIG. 13b), through which a defective graphene (129) is obtained (FIG. 13c). Chemical etching, can occur through oxidizing reaction etch, with i) acidic solutions including concentrated H₂SO₄, HNO₃, KClO₃, or their mixtures, preferably hot, or ii) alkaline solutions like hot KOH or NaOH, or iii) use of de-alloyed and nanoporous layer of Au-Ag alloy as a mask to etch graphene. The chemically modified graphene (defective graphene) may optionally be given a post-irradiation annealing (e.g., 100-600° C. for 10 minute to 24 hrs) to optimize the defective structure for bandgap changes and biomolecule attachment (as shown in FIG. 13c). Electrical lead wires and size-limiting dielectric layer (polymer or ceramic) are then added (as shown in FIG. 13d) to make the solid state electronic sensor for the attachment of a single enzyme molecule and for the interactions of template binding and nucleotide incorporation for sequencing analysis.

FIG. 13d illustrates a molecular bridge solid state sensor (124) for genome or DNA sequencing detection via change of current or other signals upon attaching or detaching of biomolecules or nucleotide incorporation. A suspended graphene device is also possible. A single enzyme molecule (126) (e.g., DNA or RNA polymerase) is attached onto a size-limited graphene bridge (128). Nucleotide monomers (130) (A, T, C, G, U etc.) are detected upon addition to double stranded DNA (132).

Non-limiting examples of chemical etching include an oxidizing reaction etch, such as with (i) acidic solutions, including concentrated H₂SO₄, HNO₃, KClO₃ or their mixtures, preferably as a hot solution; or (ii) alkaline solutions, such as KOH or NaOH, preferably as a hot solution; or (iii) by using a de-alloyed and nanoporous layer of, for example, Au-Ag alloy film (2-20 nm in thickness, sputter or evaporation deposited) as a mask to etch graphene.

Defective graphene obtained by chemical etching as exemplified in FIG. 13 may be additionally post-etch annealed (e.g., at 100-600° C. for 10 minutes to 24 hrs) for further bandgap changes and improved biomolecule attachment. Yet another embodiment for sequencing comprises a combination of carbon nanotubes and graphene onto which a single polymerase enzyme is attached for nucleotide incorporation sensing and sequencing analysis.

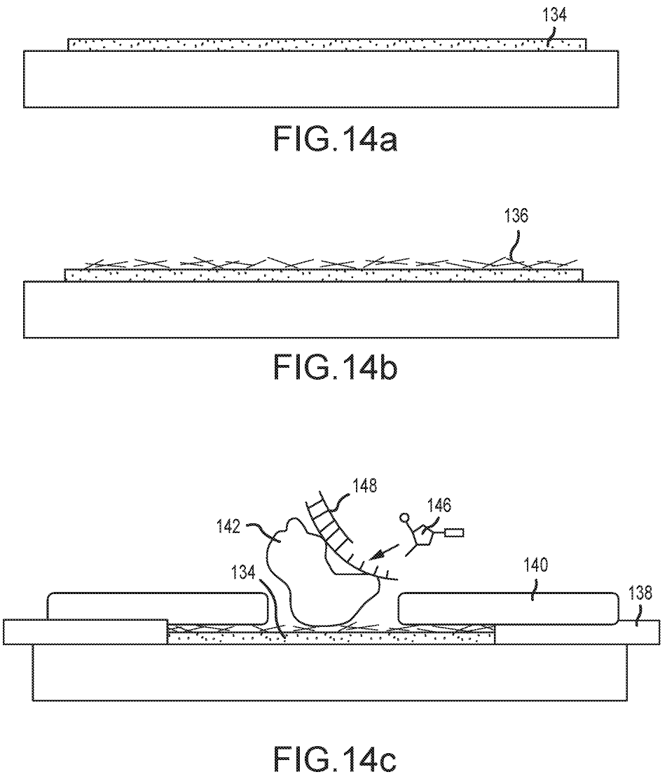

Shown in FIG. 14 is a combination structure of nanowires (e.g., carbon nanotubes, or semiconductor nanotubes, semiconductor nanowires like ZnO, semiconductor nanoribbons) adhered onto the surface to produce a defective graphene structure for enhanced molecular bridge DNA sensor. In various embodiments, the method comprises: providing a graphene layer on a substrate (as shown in FIG. 14a); adding semiconductor nanotubes or nanowires on the graphene (as shown in FIG. 14b); and adding electrical lead wires and size-limiting dielectric mask layer (polymer or ceramic) to make the solid state electronic sensor for single enzyme molecule and associated analytes attachment and sequencing analysis (as shown in FIG. 14c). On the surface of the pre-attached graphene layer (134) (as shown in FIG. 14a), randomly distributed semiconductor nanotubes or nanowires (136) are added (as shown in FIG. 14b). Electrical lead wires (138) and size-limiting dielectric mask layer (polymer or ceramic) (140) are then added (as shown in FIG. 14c) to make the solid state electronic sensor for single enzyme molecule and associated analytes attachment and sequencing analysis.

Examples of a semiconductor nanowire that can be added to the graphene are single wall carbon nanotubes such as Si nanowires, ZnO nanowires (as detailed in FIG. 14b). The nanowires are adhered to the graphene surface through van der Waals forces (as detailed in FIG. 14b). This results in a defective assembly structure for bandgap change or enhanced adhesion of biomolecules or enhanced structural integrity (as detailed in FIG. 14b).

The resulting defective graphene can be used in a molecular bridge solid state sensor for genome or DNA sequencing detection via change of current or other signals upon attaching or detaching of nucleotide or other biomolecules (as shown in FIG. 14c). As shown in FIG. 14c, a single enzyme molecule (142) (e.g. DNA or RNA polymerase) is attached onto a size-limited graphene bridge (134). Nucleotide monomers (146) (A, T, C, G, U, etc.) are detected upon incorporation into the DNA strand (148) through a change in current in the electrical lead wires (138) (Au, Pt, Pd, alloys).

The desired density of nanotubes or nanowires on graphene is at least 10³/cm², preferably at least 10⁶/cm² and even more preferably at least 10⁹/cm². The diameter of the carbon nanotubes can be in the range of about 0.8-5 nm for the case of single wall nanotubes. The semiconductor nanowires to decorate the graphene surface can be selected from Si or Ge nanowires, or doped Si or Ge nanowires, ZnO nanowires or doped ZnO nanowires by Al, N, Ga, Li, Ni, Cu. The length of the nanotubes or nanowires can be in the range of about 100 nm to 100 μm, such as for example 1-50 μm.

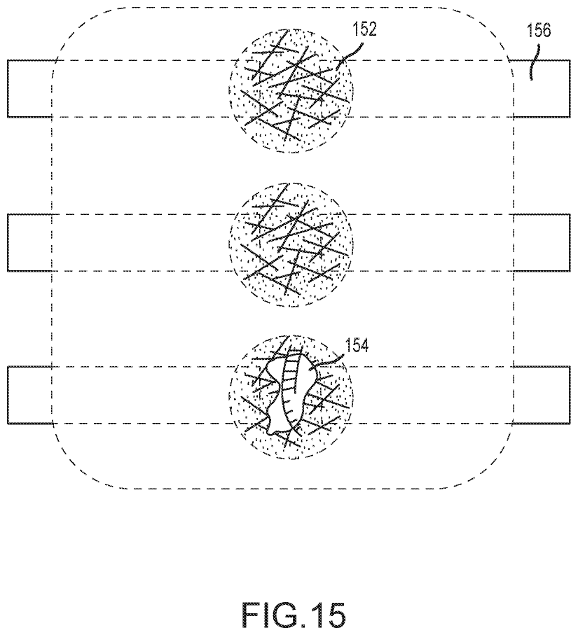

The nanotube or nanowire decorated graphene device can be assembled into a multitude of arrayed devices as illustrated in FIG. 15, which illustrates a top view of an embodiment of an array of molecular bridge sensors having a semiconductor nanotube- or nanowire-decorated graphene, having a size-limited, locally exposed geometry (pristine or preferably defective or porous graphene), with the exposed island region mask pattern defined by dielectric coating (e.g., PMMA, PDMS, other adhesion-impeding polymer layer or SiO₂ etc.) to prevent biomolecule attachment except the island areas. The sensor depicted in FIG. 15 contains multiple arrays of conducting lead wires (156) (Au, Pt, Ag, Pd, Rh, or their alloys, etc.) for signal detection with the size-limited graphene island and superimposed carbon nanotubes (CNT, preferably single wall type) or semiconductor nanotubes or nanowires. Also illustrated in FIG. 15 are units of molecular bridge sensors comprising a semiconductor nanotube- or nanowire- decorated graphene (152), with a single polymerase enzyme (154) on top. The graphene is positioned as a suspended layer or substrate-adhered layer. A single polymerase enzyme molecule placed on top of the nanotube- or nanowire-decorated graphene is also illustrated on the nanogap near the lower electrode pair in the drawing.

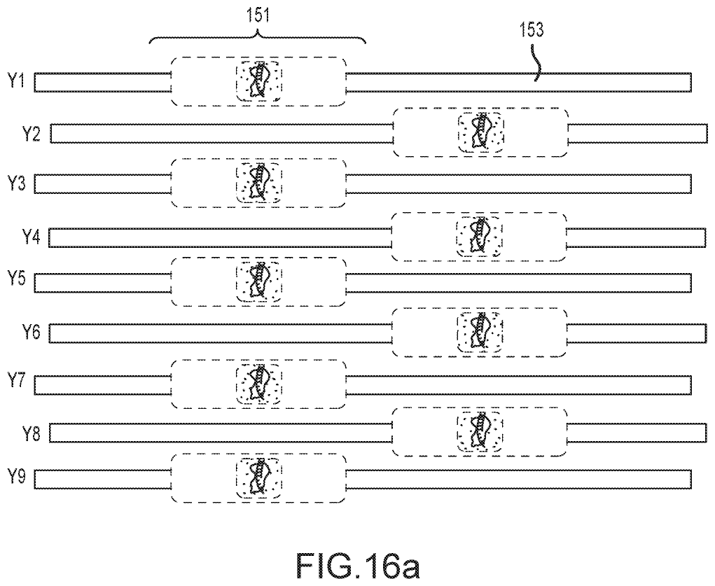

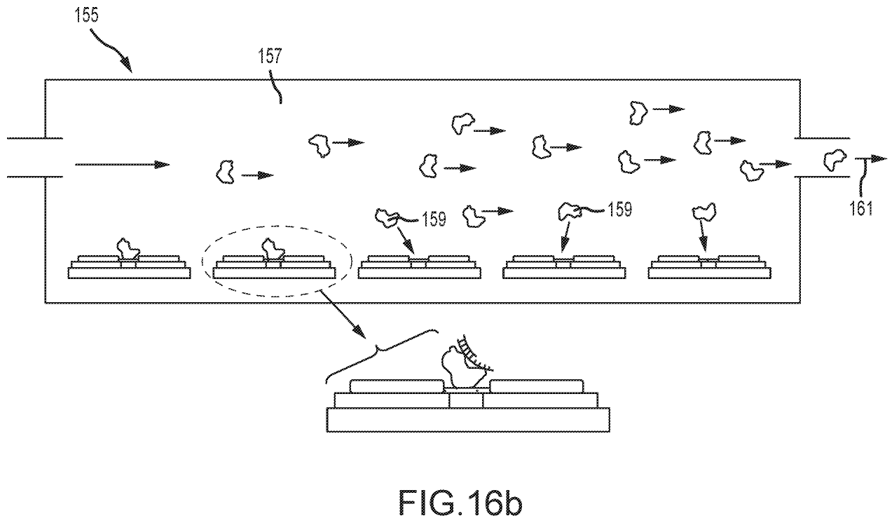

Solid state molecular sensors for a nucleic acid sequencing device comprising a combined structure of size-limited graphene-derivative material and a single molecule polymerase enzyme as described herein, and for example in FIGS. 2-7 and FIGS. 11-15 can be made into an array of at least thousands of devices, as illustrated in FIG. 16a, which illustrates graphene-containing enzyme polymerase molecular sensor (151) for label-free detection of nucleotide attachment. An Au lead wire (153) is surface insulated or coated to prevent protein adhesion. The availability of many parallel devices allows the sequencing analysis to be conducted faster, and also with more accuracy through redundant error correction and greater coverage of the target sequence or genome. Also, such a parallel processing scheme provides efficiency of scale, reduced reagent consumption and reduced handling and processing labor which thereby reduces overall system costs and the costs of sequencing a given target or whole genome.

Shown in FIG. 16b is a schematic illustration of a microfluidic reaction chamber (155) wherein the enzyme or biomolecules and associated chemical components are attached onto each of a massively parallel configured graphene-derived molecular sensor device. A phosphate-buffered saline (PBS) or other aqueous buffer solution containing enzyme or other biomolecules (157) is supplied to a microfluidic reaction chamber. Individual single enzyme molecules (159) are selectively attached with a high probability onto each of the size-limited, exposed graphene island regions on the massively parallel sensor array. The probability of single molecule attachment onto each graphene island is high because of the size-limited island configuration of the graphene, which is further enhanced because of the artificially and intentionally added, very large number of defects and more active binding sites such as the edge sites in nanoporous graphene. In some embodiments, the success ratio of a single molecule adhesion to the available graphene island locations is at least 30%, preferably at least 60%, even more preferably at least 80%.

In some embodiments, an important aspect of using graphene as the base structure in the microfluidic system is the generally desired hydrophilicity of graphene. It is well known that pristine, uncontaminated graphene is hydrophilic, while contaminated graphene (e.g., via inadvertent surface deposition grease-type hydrophobic material on the graphene surface) tends to exhibit less hydrophilic characteristics. See an article by Z. Li et al, “Effect of airborne contaminants on the wettability of supported graphene and graphite” Nature Materials 12, page 925-931 (2013). It has been demonstrated that the substrate affects the hydrophilicity of graphene, and it is also known that the hydrophilic of graphene can be noticeably improved by doping (e.g., p-type doping), or by simply applying a bias voltage. See an article by See an article by Guo Hong et al, “On the Mechanism of Hydrophilicity of Graphene”, Nano Lett. 16, page 4447-4453 (2016). In some embodiments, the deposited graphene layer is processed and kept carefully so as to minimize long-term air exposure and inadvertent collection of hydrocarbon or grease-like contaminants, so as to maintain the general hydrophilicity with a water droplet contact angle of less than 70 degrees. Also a brief application of a bias voltage of e.g., 50-100 V can be utilized if needed to enhance the adhesion of polymerase type enzyme molecules or other biomolecules onto graphene base structure.

The unused biomolecules which did not adhere to the graphene-derived islands are washed away and out of the fluidic chamber (161). The analytes to be detected, such as nucleotides (dNTPs), DNA templates, or proteins, are then supplied into the chamber to attach to the polymerase enzyme to induce electrical signals for biomolecule identification or DNA sequencing analysis.

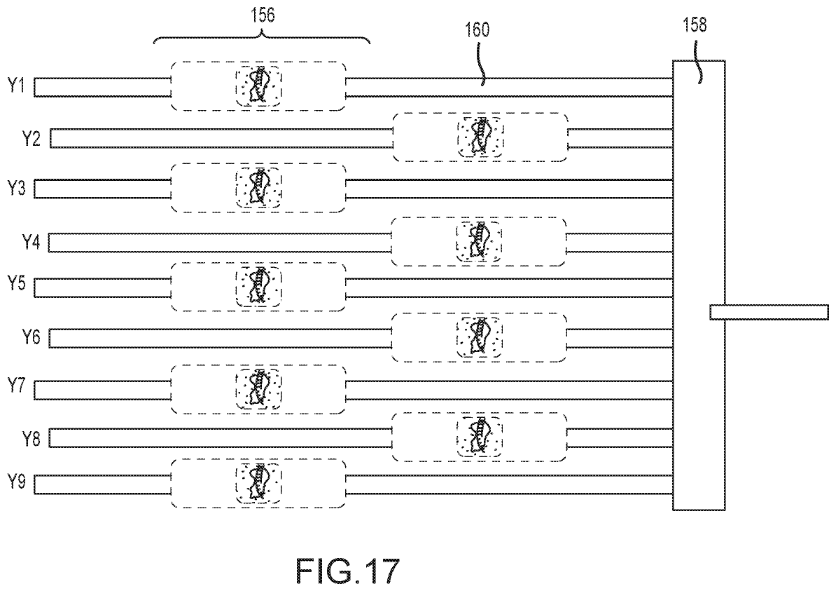

Electrical connections for many parallel devices may be complex and take up device surface area. One way of reducing the complexity and analysis time is to utilize a sequential interrogation of electrodes from the graphene-derived single enzyme molecular sensor by using a common lead wire on one side of the array, as illustrated in FIG. 17, by shorting one side of all lead wires and taking turns with the left side electrical lead wires one at a time, e.g., every millisecond, as shown in FIG. 17. Specifically, FIG. 17 illustrates a graphene-containing enzyme polymerase molecular sensor (156) for label-free detection of nucleotide attachment. An Au lead wire (160) is surface insulated or coated to prevent protein adhesion. Each of the right side electrodes are connected to a common lead wire (158). The left side electrodes are interrogated one at a time, sequentially. This method has an advantage of requiring less electronic measurements, and avoids the complications of handling many thousands of parallel signals all at once.

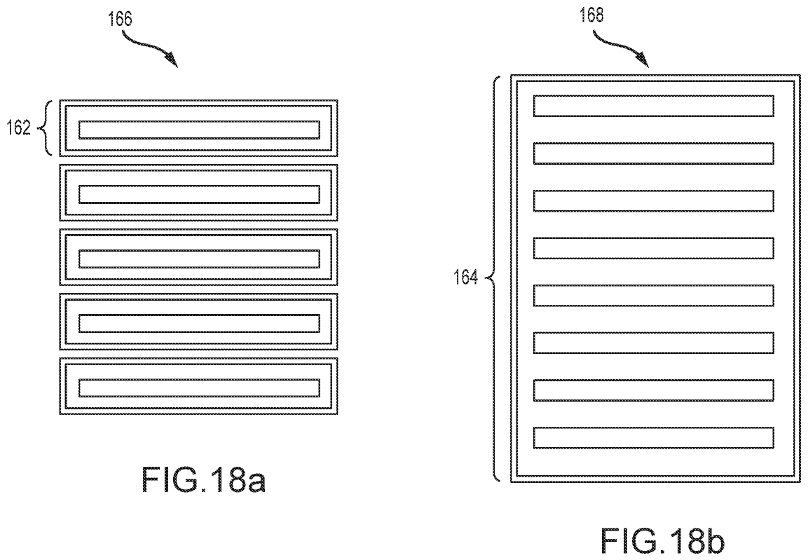

Even larger data can be obtained if the sensor device structure depicted, for example in FIG. 16 or FIG. 17 is stacked in three dimension as shown in FIG. 18, with accompanying microfluidic chambers for each stack (162) (as shown in FIG. 18a) or using one or more of common microfluidic chamber (164) (as shown in FIG. 18b). FIG. 18a illustrates a microfluidic chamber with 10-1,000 layers (166), each layer having, e.g., 100˜10,000 devices in individualized microfluidic chambers with PBS solution and nucleotide control arrangements and an electronic sensing array. FIG. 18b illustrates a microfluidic chamber containing 10 to 1,000 layers (168), each layer having , e.g., 100˜10,000 devices. Each device layer in FIGS. 18a and 18b contain a multitude of molecular sensors. In such 3D configurations, at least 10,000 to 1,000,000, and preferably 10,000,000 to 1 billion, sensor devices could be operated simultaneously for extremely rapid DNA or genome sequencing.

When the sequencing device is not in use or is being delivered or stored, it is important to make sure that the chamber is thoroughly washed and dried with a clean gas to minimize adhesion or adsorption of unwanted gas molecules, dirt or dust nanoparticles or microparticles. The sequencing device when not in use or in storage is desirably evacuated using a vacuum pump and back-filled with an inert gas such as nitrogen, argon or xenon, so as to minimize the presence of unwanted gas molecules or dirt particles.

As provided for and described herein, a polymerase is an enzyme that synthesizes long chains or polymers of nucleic acids. For example, DNA polymerase and RNA polymerase can copy a DNA or RNA template strand, respectively, using base-pairing interactions, which is utilized to assemble DNA and RNA molecules. In some embodiments, when a particular type of nucleotide or other biomolecule is attached to the enzyme polymerase biomolecule, a unique pulse current signal is generated which provides information on what type of nucleotides is being attached or detached. In some embodiments, part of the sequencing operation includes the nucleotide attachment to form a double-strand DNA associated with the polymerase enzyme.

In some embodiments, the biomolecule to be attached on the graphene includes, in addition to the enzyme molecules, various other polymers, DNA, RNA, proteins, ribozyme, aptamer or polysaccharide. In some embodiments, other single molecule functionalizations of these defective graphene structures can provide sensors for other applications areas than genome sequencing. For example, an enzyme other than polymerase can be attached, and this becomes a sensor for the activity of that enzyme. This can be used to sense the presence of the enzyme substrate, and can also be use to characterize the precise kinetics of the enzyme, for application to enzyme evolution, selection and optimization. In some embodiments, if a single molecule binding probe is attached to the defective graphene, such as a single stranded DNA or RNA oligomer hybridization probe, or an antibody against an antigen, or a protein that engages in a protein-interaction binding, this can be used to sense the binding events, and thus acts as a sensor for the presence of the binding target.

### Modified dNTPs for Enhanced Sequencing Applications of Bridge Molecules

In some embodiments, for DNA or genome sequencing applications, the bridge molecule is conjugated to a polymerase, bound with a primed single-stranded template DNA, and provided with a buffer containing nucleotides, dNTPs (deoxynucleotide triphosphates) for incorporation. In some embodiments, electric current through the bridge molecule is monitored as dNTPs are incorporated to synthesis the complementary strand of the template DNA. In the preferred embodiment, native dNTPs like A, T, C, G are used (dATP, dTTP, dCTP and dGTP).

In some embodiments, any or all of these may be replaced by corresponding modified dNTPs, having various molecular modifications that may enhance the incorporation signals, or produce signals with enhanced differences between the different base (A, C, G, T) incorporation events, for greater accuracy determining the template sequence. Such dNTP modifications could include: modifications of the base, such as 7-deaza forms, 8-bromo forms, or modification of the alpha- and beta- phosphates, such as thiolated forms or bromated forms of these phosphates, or gamma-phosphate modifications, including the addition of phosphates, such as tetra-, penta- or hexa- phosphates forms.

In some embodiments, modifications could include groups added to the terminal phosphate, and it is known that the polymerase is highly tolerant of many diverse groups added to the terminal phosphate, thus providing a large class of modified dNTPs for these purposes. The use of such modified dNTPs to enhance signals does not require any labeling of the template DNA, or any other use of labels; instead, the modified molecular forms modify the conductivity properties of the complex, and thereby directly enhance the resulting electronic detection signals. Throughout this disclosure, whenever the expression “nucleotide” is used, it refers to either the native dNTPs like A, T, C, G (i.e., dATP, dTTP, dCTP and dGTP), or collectively refers to various types modified dNTPs as described above.

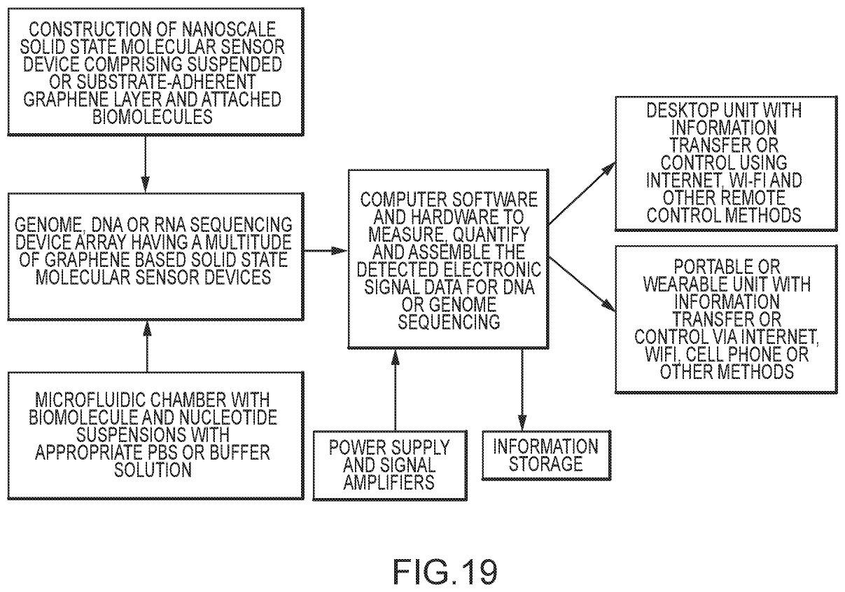

The overall genome or DNA sequencing system is described in FIG. 19 as a block diagram. Appropriate data analysis and storage software, computer systems (and if desired wireless data transfer capability (e.g. Wi-Fi)) are incorporated, and a microfluidic system is connected to the massively parallel electronic detection device array comprising the combined structure of graphene-derived material and single molecule polymerase enzyme, wherein such a system provides the DNA templates to be sequenced and supplies suitable dNTP substrates in a buffer solution. This system can be embodied in a physical format of a benchtop units, or as portable, mobile, point-of-use, hand-held or wearable units, with information transfer or control via internet, Wi-Fi, cell phone or any other convenient method that supports such distributed or mobile uses.

The nanofabrication, assembly and packaging of the devices described herein are desirably compatible with standard electronic device fabrication and assembly such as CMOS device fabrication and assembly. The electrodes and detection circuitry are preferably fabricated uses a CMOS process, with graphene-derived materials added in subsequent post processing steps, and molecular components added in subsequent self-assembly chemical reactions in suitable aqueous buffering and ionic solutions that support the self-assembly chemistry and stability and functional integrity of the biomolecular components.

In some embodiments, the DNA or RNA sequencing device comprising modified or defective graphene material derived from graphene is useful for partial or whole genome sequencing. This system is also useful for diagnosis of diseases with a genetic component, by coupling this system with suitable sample intake procedures and procedures for the interpretation of sequence data and generation of a final diagnostic report. Such diseases include, for example, heritable genetic diseases such as (monogenic) sickle cell anemia, cystic fibrosis or congenital deafness or such as (polygenic) diabetes or obesity or Alzheimer's, and for diseases arising from somatic DNA mutations, such as cancer and diseases of tissue aging. This also includes diagnosis diseases due to viral or microbial changes in the bodily microbiomes, such as during viral infections, sepsis, or disruption of normal gut microbiome bacterial populations, which can be monitored through the observation of viral or bacterial genome sequences in suitably obtained samples of relevant bodily fluids or tissues. The genome sequencing system can be operated for these purposes as a desktop unit, a portable unit, a point-of-care unit, or a wearable monitoring unit.

### EXAMPLES

### Example 1: Method of Producing a Molecular Bridge of a DNA or RNA Sequencing Sensor

A conducting electrode pair (10) (Au, Pt, Ag, Pd, Rh, or their alloys) that contains a nanogap (12) of between 2 and 20 nm is deposited on a dielectric substrate (14) (SiO₂, Al₂O₃, etc.) (FIG. 2a). A suspended graphene layer (16) (regular or intentionally made defective) is then added (FIG. 2b). The graphene sheet is then coated with a protective, size-limiting dielectric coating (18) (e.g., polymer layer like PMMA or ceramic layer like SiO₂). The graphene bridge is limited (e.g., 2-20 nm) for preferably a single biomolecule (20) (e.g., polymerase enzyme) attachment (FIG. 2c). This dimensional limitation allows a single enzyme biomolecule (20) (e.g., DNA or RNA polymerase) to attach onto the size-limited graphene bridge, which allows the nucleotide monomer (22) (e.g., A, T, C, G, U, etc.) to be detected on polymerase reaction (FIG. 2d). The monomer is detected through a change of current pulse upon attachment of the monomer to the DNA.

### Example 2:Methods of Graphene Bridge Placement

Suspended Graphene Bridge: The suspended graphene bridge (24) is attached/affixed by Van der Waals force and a dielectric coating (25) (FIG. 3a). A single enzyme molecule (30) (e.g., DNA or RNA polymerase) is attached onto the size limited graphene bridge (FIG. 3a). A nucleotide monomer (32) (A, T, C, G, U, etc.) is detected on polymerase reaction (FIG. 3a). The monomer is detected through a change of current pulse upon attachment of the monomer to the double stranded DNA (FIG. 3a).

Substrate Adhered Graphene Bridge: The substrate-adhered graphene bridge (38) is affixed onto the dielectric substrate, for example, by a Van der Waals force and dielectric coating. A single enzyme molecule (39) (e.g., DNA or RNA polymerase is attached onto the size limited graphene bridge (FIG. 3b). A nucleotide monomer (33) (A, T, C, G, U, etc.) is detected on polymerase reaction (FIG. 3b). The monomer is detected through a change of current pulse upon attachment of the monomer to the double stranded DNA (FIG. 3b).

As compared to the free-standing suspended graphene bridge, the substrate-adhered graphene (as detailed in FIG. 3b) is mechanically more robust with a less probability of fracture and other contaminations. However, the strong adhesion to the substrate could affect the electronic and thermal expansion/contraction properties and associated mechanical strain aspects. Therefore, careful engineering manipulations in terms of processing and specific details of structural configurations are necessary in order to optimize the graphene performance.

### Example 3: Method of Bonding of an Enzyme Molecule to the Graphene via the Aid of Functional Groups

An enzyme molecule (46) (e.g., DNA or RNA polymerase) is connected to a suspended graphene bridge (48) or a substrate-adhered graphene bridge (58) via a linker (50) that contains functionalization groups that link the enzyme molecule to the graphene (FIGS. 4a and 4b). A nucleotide monomer (54) (A, T, C, G, U etc.) is detected upon polymerase chain reaction (FIGS. 4a and 4b). The monomer is detected through a change in current pulse of the conducting electrodes and lead wires (Au, Pt, Ag, Pd, Rh, or their alloys, etc.) upon attachment of the monomer to the DNA (FIGS. 4a and 4b).

### Example 4: Method of a Molecular Bridge Solid State Sensor using a Nanogapped Graphene Derivative

This molecular bridge has a split-nanogapped graphene region (76) and (78) as a well-adhered substrate for tunnel junction construction (FIG. 7a). There is a dielectric mask layer (77) (e.g., PMMA, PDMS, SiO₂, or other adhesion-impending polymer layer) that prevents biomolecule attachment (FIGS. 7b-7d). The single enzyme molecule (82) (e.g., DNA or RNA polymerase) attaches onto a size-limited graphene bridge on the nanogap in a tunnel junction configuration (FIG. 7a). Attachment of a nucleotide monomer (84) to the DNA polymerase (82) results in change of current pulse in the conducting electrodes and lead wires (Au, Pt, Ag, Pd, Rh, or their alloys, etc.) (FIG. 7a).

Example 5: Method of Graphene Synthesis by Chemical Vapor Deposition (CVD) using a Block Copolymer

The graphene (160) is deposited on a Cu or Ni foil (162) (FIG. 8a). After the Cu or Ni substrate is etched away using, for example, FeCl₃, the graphene (160) is lifted up into a SiO₂ substrate (164) or an SiO₂-coated substrate (optionally with Au electrode pair on the substrate) (FIG. 8b). This results in floating graphene in either H₂O or alcohol (166) (FIG. 8b). The graphene is then covered by a thin layer of SiO₂ (168) and a block copolymer such as PS-b-P4VP (170) (FIG. 8c). The PS-b-P4VP block copolymer is then developed into a nanoscale two-phase structure through annealing (FIG. 8d). Reactive ion etching is then used to etch away one of the phases (FIG. 8d). O₂ plasma is used to etch away graphene in the exposed holes (FIG. 8e). SiO₂ is then removed to create the nano-patterned graphene (172) (FIG. 8f). An example of nanoporous graphene as a result of such processing is shown in FIGS. 8g and 8h.

### Example 6: Method of Graphene Synthesis by Chemical Vapor Deposition (CVD) using an AAO Membrane

The graphene (180) is deposited on a Cu or Ni foil (182) (FIG. 9a). After the Cu or Ni substrate is etched away using, for example, FeCl₃, the graphene (180) is lifted up into a SiO₂ substrate or an SiO₂-coated substrate (184) (optionally with Au electrode pair on the substrate) (FIG. 9b). This results in floating graphene in either H₂O or alcohol (186) (FIG. 9b). An AAO membrane (188) is then synthesized by anodization of AI foil and the AI matrix is dissolved away (FIG. 9c). The AAO membrane (188) is then floated in a liquid such as H₂O or alcohol. The graphene substrate is lifted up to pick up the AAO membrane (FIG. 9d). A dry coating of AAO is added and reactive ion etching is performed through the AAO masked holes (190) (FIG. 9e). The AAO is removed to create the nano patterned graphene (192) (FIG. 9f).

### Example 7: Method of Generating Defective Graphene by Using Incompletely Deposited Thin Film Mask on Graphene with Plasma or Chemical Etching through the Mask

A graphene layer (87) is deposited on a removable dielectric substrate (89) (SiO₂, Al₂O₃, etc.) or a metallic substrate, either by chemical vapor deposition (CVD) or by a transfer of pre-made graphene sheet floating in aqueous or alcohol-containing solution onto a substrate by lifting up and drying (FIG. 10a ). A thin film mask layer (88) is then deposited on the top surface of graphene with a thickness of less than 5 nm, in an incomplete manner so that some nano regions are still not completely filled and nano-pinholes are present (FIG. 10a). The thin film is deposited by sputtering although other method of deposition such as evaporation, pulsed laser deposition or chemical deposition can also be utilized. The material for the thin film mask is selected from a metallic layer such as Ni, Co, Cu, Mo, or W. Other layers such as a SiO₂ type ceramic layer or polytetrafluoroethylene (PTFE) type polymer layer can also be utilized for sputter deposition.

A relatively short period deposition of the thin film layer is performed so as to produce pinhole-containing, thin (less than 5 nm thick) film time, with the pinhole size in the range of 0.2-2 nm average diameter, and a density of 10⁵/cm² or higher. An optional post-sputtering can also be applied to consolidate the pinholes to a more uniform sizes. In order to increase the pinhole type defects in sputter deposition, the thin film mask deposition is carried out preferentially at a lower temperature, preferably near or below room temperature. The graphene underneath the thin film mask is then etched away (91) through the mask defects using either oxygen plasma (other types of plasma etching such as by using argon, N₂, NH₃ plasma can also be utilized) or using chemical etching with an alkaline solutions like hot KOH or NaOH. Acidic solutions of concentrated H₂SO₄, HNO₃, KClO₃ or their mixtures can also be utilized for graphene etching (FIG. 10e). Once the nanopinhole defects are prepared in the graphene, the mask material is removed by selective chemical etching or plasma etching. Metallic thin film masks are easily etched away by dilute acids, while SiO₂ type masks are readily removed by a BOE (buffered oxide etch) solution etching having a 6:1 volume ratio of 40% NH₄F in water to 49% HF. The mask layer can also be etched away by plasma etching or ion beam etching. If NaCl or other salt type material is sputter deposited and employed as the mask layer, it can be dissolved away by water or solvents. Polymer mask films can be removed by solvent or by plasma etching.

The defective graphene created by using such a thin film mask approach can be utilized for genome sequencing if they are prepared directly on a support (sequencing structure base, or split substrates for graphene suspension) by micro- or nano-patterning to have a limited-size to enable a single molecule polymerase attachment, followed by nucleotide attachment analysis.

Alternatively, the defective graphene created by using such a thin film mask approach on a general substrate or dissolvable substrate can be released from the substrate by etching away, floated in a aqueous or alcohol-containing solution, picked up by the genome sequencing base structure and micro patterned into desired geometry. A dielectric masking layer is placed on the defective graphene surface with an opening which is size-limited so as to allow only a single enzyme biomolecule to attach onto the exposed graphene surface in a microfluidic system into which the DNA or genome sequencing structure is inserted. An electronic measurement and computer analysis are performed on attachment or detachment of a nucleotide monomer, a protein, a DNA segment, or other biomolecular component onto the enzyme polymerase molecule one at a time, which is then monitored to measure an electrical signal pulse and determine the specific nature of the nucleotide or biomolecules being attached.

### Example 8: Method of Generating Perforated Defective Graphene using Nanoparticles

A graphene layer (90) is deposited on a dielectric substrate (92) (SiO₂, A1₂0₃, etc.) (FIG. 11a). Through spraying, liquid-dispersion-coated and dried, or solid film deposition, nanoparticles (94) are coated onto the graphene (FIG. 11b). The nanoparticles are balled-up by annealing into Ag, Ni, or Cu islands (94) (FIG. 11b). After reacting with the nanoparticles, perforated defective graphene (95) is created (FIG. 11c). Etching away of the reacted nanoparticles induces perforated graphene for bandgap opening and easier biomolecule attachment (FIG. 11c). This defective graphene can then be used as part of a molecular bridge solid state sensor for genome or DNA sequencing detection (FIG. 11d).

### Example 9: Method of Generating Defective Graphene using Irradiation

A graphene layer (115) is deposited on a dielectric substrate (117) (SiO₂, Al₂O₃, etc.) (FIG. 12a). The graphene layer is then subjected to broad beam irradiation with ion implantation (111) (e.g., with N, C, F, O, H, Ar, Ni, Ti, Mn, Fe, Cu, AI and other species), plasma RIE, defocused neutron beam, laser beam, or electron beam (FIG. 12b). After the irradiation, the graphene can, optionally, be post-irradiated annealed at a temperature between 100 and 600° C., to induce point defects, interstitials or agglomerated nanoparticle, or nanopore defects (113) for bandgap change and easier biomolecule attachment (FIG. 12c).

This defective graphene can then be used as part of a molecular bridge solid state sensor (110) for genome or DNA sequencing (FIG. 12d).

### Example 10: Method of Generating Defective Graphene through Chemical Etching

A graphene layer (123) is deposited on a dielectric substrate (125) (SiO₂, Al₂O₃, etc.) (FIG. 13a). The graphene layer is then subjected to chemical etching through oxidizing reaction etch, with (i) acidic solutions including concentrated H₂SO₄, HNO₃, KClO₃, or other mixture, preferably hot, (ii) alkaline solutions like hot KOH or NaOH, or (iii) use of a de-alloyed and nanoporous layer of Au-Ag alloy as a mask to etch graphene (127) (FIG. 13b). After chemical etching the graphene is optionally post-irradiated annealed at 100-600° C. (129) for bandgap change and easier biomolecule attachment (FIG. 13c). This defective graphene can then be used as part of a molecular bridge solid state sensor for genome or DNA sequencing (FIG. 13d).

### Example 11: Method of Generating Defective Graphene through Adhering Nanowires to the Graphene

A graphene layer (134) is deposited on a dielectric substrate (SiO₂, A1₂0₃, etc.) (FIG. 14a). Semiconductor nanowires such as single wall carbon nanotubes, e.g. Si nanowires, ZnO nanowires (136) are adhered onto the graphene surface through Van der Waals forces (FIG. 14b). This creates a defective assembly structure for bandgap change or enhanced adhesion of biomolecules or enhanced structural integrity (FIG. 14c). This defective graphene can then be used as part of a molecular bridge solid state sensor for genome or DNA sequencing (FIG. 14d).

The embodiments and examples described above are only to exemplify the embodiments disclosed herein but not to limit the scope of the embodiments. Any equivalent modification or variation according to the spirit of the embodiments disclosed herein is to be also included within the scope of the embodiments.

What is claimed is:

1. A sequencing device structure comprising:
an array of metallic conducting electrode pairs, each electrode pair defining a bridging source and drain arrangement separated by a nanogap, the electrode pairs deposited and patterned on a dielectric substrate;
a graphene layer deposited onto each electrode pair bridging the source and drain electrodes in each pair, wherein each electrode pair is in electrical isolation from each other; and
a dielectric masking layer contacting the graphene layer, the masking layer having an opening exposing a portion of the graphene layer directly over each nanogap, wherein each opening is dimensioned in size to accommodate at least one polymerase enzyme molecule.

2. The sequencing device structure of claim 1, further comprising at least one polymerase enzyme molecule bonded to the exposed portion of the graphene layer through the opening.

3. The sequencing device structure of claim 2, further comprising a microfluidic system in fluid combination with the sequencing device structure to provide the at least one polymerase enzyme molecule.

4. The sequencing device structure of claim 1, wherein each electrode pair comprises at least one of Au, Pt, Ag, Pd, Rh, or their alloys.

5. The sequencing device structure of claim 1, wherein the nanogap is about 2 nm to about 20 nm in length.

6. The sequencing device structure of claim 1, wherein the graphene layer comprises defective graphene.

7. The sequencing device structure of claim 6, wherein the defective graphene comprises a linear nano-ribbon parallel array, a patterned shape nano-ribbon array, disturbed lattice defects, nanoporous defects, or compositionally doped defects.

8. The sequencing device structure of claim 7, wherein the defective graphene comprises disturbed lattice defects having a defect density of at least about 10⁵/cm².

9. The sequencing device structure of claim 7, wherein the defective graphene comprises nanoporous defects having a diameter of at least about 2 nm with a defect density of at least about 10³/cm².

10. The sequencing device structure of claim 7, wherein the defective graphene has a bandgap opened to a value of at least 0.2 eV.

11. The sequencing device structure of claim 1, wherein the defective graphene comprises elongated nanowires that are selected from carbon nanotubes and semiconductor nanowires of doped Si, Ge, or ZnO.

12. The sequencing device structure of claim 1, wherein the graphene layer is positioned on each electrode pair such that the graphene layer does not contact the substrate at each nanogap.

13. The sequencing device structure of claim 2, wherein the bonding of the at least one polymerase enzyme to the graphene layer comprises van der Waals interactions.

14. The sequencing device structure of claim 2, wherein the bonding between the polymerase enzyme molecule and the graphene layer comprises a bifunctional linker bonded at one end of the linker to the graphene layer by pi-stacking to a pyrene group.

15. The sequencing device structure of claim 14, wherein the bifunctional linker bonds to the polymerase enzyme molecule through functional binding pairs selected from streptavidin-biotin pair, mercaptocarbonic acid [HS-(CH₂)n-COOH, n=1−15] pairs, thiol-alkyne pair, COOH-NH₂ functional group pairs, thiol-maleimide pair, cysteine-maleimide pair, silanization linkage pairings using mercaptosilane compounds, an NHS (N-hydroxysuccinimide) ester-amine pair, an antigen-antibody pair, or a click chemistry pair.

16. The sequencing device structure of claim 1, further comprising a gate electrode that is parallel to each electrode pair defining a bridging source and drain arrangement.

17. The sequencing device structure of claim 1, further comprising a gate electrode that is perpendicular to the nanogap spacing between each electrode pair defining a bridging source and drain arrangement.

18. A method of fabricating a sequencing device structure comprising:
depositing and patterning an array of metallic conducting electrode pairs on a dielectric substrate, each electrode pair defining a source and drain arrangement separated by a nanogap;
depositing a graphene layer over each metallic conducting electrode pair; and
nanopatterning a dielectric masking layer on the graphene layer.

19. The method of claim 18, further comprising processing the graphene layer to obtain a defective graphene material.

20. The method of claim 19, wherein the processing comprises nanopatterning the graphene layer into a linear nano-ribbon parallel array or a patterned shape nano-ribbon array, introducing disturbed lattice defects, or providing nanoporous defects.
